(19) United States
(12) Patent Application Publication
(10) Pub. No.: US 2011/0009760 A1
(43) Pub. Date: Jan. 13, 2011

(54) Hospital Readmission Alert for Heart Failure Patients

(76) Inventors: Yi Zhang, Plymouth, MN (US); Viktoria A. Averina, Roseville, MN (US)

Correspondence Address: HOLLINGSWORTH & FUNK, LLC;Suite 320, 8500 Normandale Lake Blvd., Minneapolis, MN, 55437, US

(21) Appl. No.: 12/787,789

(22) Filed: May 26, 2010

Related U.S. Application Data
(60) Provisional application No. 61/224,721, filed on Jul. 10, 2009.

(51) Int. Cl. A61B 5/08

(52) U.S. Cl. 600/529

(57) ABSTRACT

Physiologic data is collected for a heart failure patient during hospitalization and after discharge of the heart failure patient from a hospital, and analyzed for a change in the collected physiologic data. Deterioration of the patient's heart failure status is detected based at least in part on detecting an acute deleterious change in the collected physiologic data indicative of an abnormality in one or more physiologic parameters represented by the physiologic data. A readmission alert is generated in response to the acute deleterious change in the collected physiologic data. The readmission alert indicates that further deterioration in the patient's heart failure status may require readmission of the patient to the hospital.

### RELATED APPLICATIONS

This application claims the benefit of Provisional Patent Application Ser. No. 61/224,721, filed on Jul. 10, 2009, to which priority is claimed pursuant to 35 U.S.C. §119(e), and is related to U.S. patent application Ser. Nos. 11/300,675 filed on Dec. 14, 2005, 61/011,912 filed on Jan. 22, 2008, Ser. No. 12/356,289 filed on Jan. 20, 2009, and 61/224,719 filed on Jul. 10, 2009, which are hereby incorporated herein by reference in their respective entirety.

### FIELD OF THE INVENTION

The present invention relates generally to systems and methods for assessing a patient's heart failure status and, more particularly, to systems and methods for detecting one or more patient conditions that are indicative or predictive of a need to readmit a patient to a hospital after a clinical heart failure event has occurred.

### BACKGROUND OF THE INVENTION

The human body functions through a number of interdependent physiological systems controlled through various mechanical, electrical, and chemical processes. The metabolic state of the body is constantly changing. For example, as exercise level increases, the body consumes more oxygen and gives off more carbon dioxide. The cardiac and pulmonary systems maintain appropriate blood gas levels by making adjustments that bring more oxygen into the system and dispel more carbon dioxide. The cardiovascular system transports blood gases to and from the body tissues. The respiratory system, through the breathing mechanism, performs the function of exchanging these gases with the external environment. Together, the cardiac and respiratory systems form a larger anatomical and functional unit denoted the cardiopulmonary system.

Various disorders that affect the cardiovascular system may also impact respiration. For example, heart failure is an abnormality of cardiac function that causes cardiac output to fall below a level adequate to meet the metabolic demand of peripheral tissues. Heart failure (HF) is usually referred to as congestive heart failure due to the accompanying venous and pulmonary congestion. Heart failure may have a variety of underlying causes, including ischemic heart disease (coronary artery disease), hypertension (high blood pressure), and diabetes, among others.

Various types of disordered respiration are associated with HF. Respiration rate is linked to the patient's physical condition and is indicative of the patient's disease or health state. Clinical data collected in the ambulatory setting has demonstrated a statistically significant difference between respiration rate distributions from healthy subjects as compared to heart failure patients.

Rapid shallow breathing is one of the cardinal signs of heart failure. When the patient spends more time at higher respiration rates, this is indicative of a worsening of their HF status. The appearance of rapid, shallow breathing in a heart failure patient is often secondary to increased pulmonary edema, and can indicate a worsening of patient status. An abnormally high respiration rate thus can be an indicator of HF decompensation.

Symptoms of dyspnea are among the primary reasons why many heart failure patients return to the hospital during a HF decompensation episode. It is estimated that nearly one million hospital admissions for acute decompensated heart failure occur in the United States each year, which is almost double the number admitted 15 years ago. The re-hospitalization rates during the 6 months following discharge are as much at 50%. Nearly 2% of all hospital admissions in the United States are for decompensated heart failure patients, and heart failure is the most frequent cause of hospitalization in patients older than 65 years. The average duration of hospitalization is about 6 days. Despite aggressive therapies, hospital admissions for HF continue to increase, reflecting the prevalence of this malady.

Because of the complex interactions between the cardiovascular, pulmonary, and other physiological systems, as well as the need for early detection of various diseases and disorders, an effective approach to monitoring and early diagnosis is needed. Accurately characterizing patient respiration aids in monitoring and diagnosing respiration-related diseases or disorders. Evaluating patient respiration information may allow an early intervention, preventing serious decompensation and hospitalization.

### SUMMARY OF THE INVENTION

The present invention is directed to systems and methods for assessing a patient's heart failure status, and determining or predicting whether the patient may require re-hospitalization due to worsening of the patient's post-hospitalization heart failure status. Embodiments of the present invention are directed to systems and methods for assessing one or more physiologic parameters of a heart failure patient, and detecting early onset of worsening of the patient's heart failure after hospital discharge. Embodiments of the present invention are directed to systems and methods for generating a readmission alert in response to detecting acute worsening of a patient's heart failure following hospitalization within a time frame that allows for physician intervention and avoidance of hospital readmission of the patient.

Embodiments of the present invention involve collecting physiologic data for a heart failure patient during hospitalization and after discharge of the heart failure patient from a hospital, and analyzing the collected physiologic data for a change in the collected physiologic data. Deterioration of the patient's heart failure status is detected based at least in part on detecting an acute deleterious change in the collected physiologic data indicative of an abnormality in one or more physiologic parameters represented by the physiologic data. A readmission alert is generated in response to the acute deleterious change in the collected physiologic data. The readmission alert indicates that deterioration in the patient's heart failure status may require readmission of the patient to the hospital.

Various embodiments of the present invention involve collecting respiration data for a heart failure patient during hospitalization and after discharge of the heart failure patient from a hospital, and measuring, using the collected respiration data, a median respiration rate (MedRR) and a maximum respiration rate (MaxRR). The MedRR and MaxRR are analyzed for a change in the MedRR and MaxRR. Deterioration of the patient's heart failure status is detected based at least in part on detecting an acute deleterious change in the MedRR and MaxRR indicative of an abnormality in MedRR and MaxRR. A readmission alert is generated in response to detecting the acute deleterious change in the MedRR and MaxRR.

Other embodiments are directed to medical systems that implement various readmission alert processes disclosed herein. Embodiments of the present invention are directed to medical systems that include a respiration sensor configured to generate a signal indicative of patient respiration and respiration information circuitry coupled to the respiration sensor. The respiration information circuitry is configured to collect respiration data for a heart failure patient and to measure a median respiration rate (MedRR) and a maximum respiration rate (MaxRR) using the collected respiration data. A processor is coupled to the respiration information circuitry.

The processor executes program instructions that configure the processor to cause the respiration information circuitry to collect the respiration data for the heart failure patient during hospitalization and after discharge of the heart failure patient from a hospital, analyze the MedRR and MaxRR for a change in the MedRR and MaxRR, and detect deterioration of the patient's heart failure status based at least in part on detecting an acute deleterious change in the MedRR and MaxRR indicative of an abnormality in MedRR and MaxRR. An output device is coupled to the processor and configured to generate a readmission alert in response to detection of the acute deleterious change in the MedRR and MaxRR.

Further embodiments are directed to medical systems that include at least one physiologic sensor configured to generate a signal indicative of a physiologic parameter of the patient, and physiologic information circuitry coupled to the physiologic sensor. The physiologic information circuitry is configured to collect physiologic data for a heart failure patient. A processor is coupled to the physiologic information circuitry.

The processor executes program instructions that configure the processor to cause the physiologic information circuitry to collect the physiologic data for the heart failure patient during hospitalization and after discharge of the heart failure patient from a hospital, analyze the collected physiologic data for a change in the collected physiologic data, and detect deterioration of the patient's heart failure status based at least in part on detecting an acute deleterious change in the collected physiologic data indicative of an abnormality in the collected physiologic data. An output device is coupled to the processor and configured to generate a readmission alert in response to detection of the acute deleterious change in the collected physiologic data.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIG. 1 is a block diagram of a system for implementing various processes of physiologic data trending and hospital readmission alert generation in accordance with embodiments of the invention;

FIGS. 2 and 3 are flow charts illustrating various processes for determining acute worsening of a patient's heart failure status following hospitalization for a clinical HF event and generating a readmission alert responsive to same in accordance with various embodiments of the present invention;

FIGS. 4A-4D show representative plots of different respiration rate metrics and patient activity data associated with methodologies for detecting acute worsening of patient heart failure and generating a readmission alert responsive to same in accordance with embodiments of the present invention;

FIG. 5 graphically illustrates an improvement in detecting acute worsening of patient heart failure status when using both MaxRR and MedRR metrics in accordance with embodiments of the invention;

FIGS. 6 and 7 are charts that illustrates relationships between various events in connection with detecting acute worsening of a patient's heart failure and generation of a hospital readmission alert in accordance with embodiments of the present invention;

FIGS. 8-10 are flow charts illustrating various processes for determining worsening of a patient's heart failure status following hospitalization for a clinical HF event and generating a readmission alert if it is likely that the heart failure patient will require readmission because of worsening heart failure in accordance with embodiments of the present invention;

FIG. 11 illustrates respiration and activity data for a heart failure patient that required readmission to the hospital relatively soon after HF treatment and discharge from the hospital;

FIG. 12 illustrates respiration and activity data for a heart failure patient that did not require readmission to the hospital after HF treatment and discharge from the hospital;

FIG. 13 is a flow chart showing various processes of respiration rate trending that may be implemented as part of a readmission alert algorithm in accordance with embodiments of the invention;

FIG. 14 is a flow chart that shows various processes for generating and using daily respiration metrics as part of a readmission alert algorithm in accordance with embodiments of the invention;

FIGS. 15 and 16 illustrate an implementation for determining respiration metrics including daily maximum respiration rate, daily median respiration rate, and daily minimum respiration rate as part of a readmission alert algorithm in accordance with embodiments of the invention;

FIG. 17 illustrates a partial view of a patient implantable medical device that may be used to implement processes for detecting acute worsening of a patient's heart failure and generating a readmission alert in response thereto in accordance with embodiments of the invention;

FIG. 18 is a block diagram of a medical system that may implement various diagnostic, alert, and/or therapy processes in accordance with various embodiments; and

FIG. 19 is a block diagram of one embodiment of a medical system that may be configured to implement respiration rate trending and readmission alert processes in accordance with various embodiments of the present invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings, which form a part hereof. The specification and drawings show, by way of illustration, various embodiments in which the invention may be practiced. It is to be understood that other embodiments may be utilized, and structural and functional changes may be made without departing from the scope of the present invention.

Systems, devices or methods according to the present invention may include one or more of the features, structures, methods, or combinations thereof described hereinbelow. For example, a device or system may be implemented to include one or more of the advantageous features and/or processes described below. It is intended that such device or system need not include all of the features described herein, but may be implemented to include selected features that provide for useful structures and/or functionality. Such a device or system may be implemented to provide a variety of therapeutic or diagnostic functions.

Physiological sensors, which may be used with external and/or implantable devices, provide opportunity for collection of patient data which may be analyzed to develop trends of patient status. These trends allow a physician to assess changes in patient health, to analyze the effects of therapy, and/or to track the progression and/or regression of a disease. Changes in respiration, for example, may be caused by various patient conditions. Causes of tachypnea (fast respiration rate), by way of example, may include various factors including exertion, fever, pain, anemia, obesity, pneumonia, pneumothorax, acute respiratory distress, heart failure, hyperthyroidism, abdominal distention, respiratory muscle paralysis, chronic obstructive pulmonary disease, and/or other conditions.

Information developed from physiologic parameter data, such as respiration data, in accordance with embodiments of the present invention provides for enhanced patient detection, monitoring, and/or therapy management, particularly when the status of a patient is in decline. Embodiments of the invention are directed to systems and methodologies for detecting one or more patient conditions that are indicative or predictive of the possible need to readmit a patient to a hospital after a clinical event has occurred. For example, systems and methodologies of the present invention provide for detecting one or more patient conditions that are indicative or predictive of the possible need to readmit a heart failure patent to a hospital subsequent to discharging the patient from the hospital after a clinical HF event. Embodiments of the present invention provide a tool that can help clinicians better stratify patients and allocate their limited resources more effectively.

Heart failure readmission rates remain high despite of advancements in heart failure treatment and diagnostics. A recent study indicates that a large percentage of heart failure patients require readmission within 60-90 days following hospital discharge due to a previous clinical HF event. It is believed that a methodology that provides for early detection of a heart failure patient's likelihood for readmission following a previous clinical HF event would be valuable.

Systems and methods of the present invention advantageously provide for early physician intervention and treatment of a patient's worsening HF condition. Early detection and treatment of a patient's worsening heart failure can significantly reduce the rate of heart failure patient readmission and the high cost of heart failure patient readmission. Techniques of the present invention can determine or estimate the likelihood of readmission for a particular heart failure patient, and may further determine or estimate the rate of readmission for a population of heart failure patients. Various embodiments provide for adjusting the detection processes so as to change the relationship between sensitivity and specificity. For example, adjustment may be made to accept more false positives in trade for a higher sensitivity for the HF readmission detection.

In various embodiments of the invention, analysis of one or more physiologic parameters of a patient provides for detection of the patient's heart failure status, particularly after discharge of a heart failure patient from the hospital. Early detection of worsening of the patient's heart failure status may be detected by analysis of trend data developed for the physiologic parameter(s). Analysis of physiologic parameter data of a heart failure patient preferably triggers a readmission alert indicating an acute deleterious change in the patient status and/or reduced effectiveness of therapy (e.g., pharmacological or cardiac stimulation therapy) delivered to the patient who has been discharged from the hospital for treatment of a prior HF event.

A readmission alert is preferably generated in response to detecting an acute deleterious change in a heart failure patient's physiologic parameter data following hospital discharge. The readmission alert preferably indicates that further deterioration in the patient's heart failure status may require readmission of the patient to the hospital. The readmission alert is communicated to a device or system capable of distributing the alert to a clinician or patient advocate, allowing for timely interventional therapy and/or adjustment of lifestyle (e.g., diet), and avoidance of readmission of the heart failure patient to the hospital.

In various embodiments of the invention, analysis of the patient's respiration, which may be used alone or in combination with other physiological information, provides for detection of early onset of worsening of the patient's heart failure status, particularly after discharge of a heart failure patient from the hospital. Early onset of worsening of the patient's heart failure status following patient discharge from the hospital may be detected by analysis of the patient's daily respiratory rate trend, such as by detecting an acute deleterious change in one or more respiration metrics. Detection of early onset of worsening of the patient's heart failure status may be enhanced by analysis of the patient's daily respiratory rate trend and activity level of the patient.

In various embodiments of the invention, analysis of the patient's respiration, which may be used alone or in combination with other physiological information, triggers an alert indicating a change in the patient status and/or effectiveness of therapy (e.g., pharmacological or cardiac stimulation therapy) delivered to the patient. In various embodiment, analysis of the patient's respiration alone or in combination with patient activity level triggers an alert indicating an acute deleterious change in the patient's tachypnea status. Analysis of the patient's respiration, alone or in combination with patient activity level, triggers an alert indicating detection of an acute deleterious change in the patient's tachypnea status indicative of an abnormality in the patient's respiration metrics. A readmission alert is preferably generated in response to detecting the acute deleterious change in patient respiration metrics. The readmission alert preferably indicates that the deterioration in the patient's heart failure status may require readmission of the patient to the hospital. The readmission alert is communicated to a device or system capable of distributing the alert to a clinician or patient advocate.

The systems and processes described herein may be particularly effective in monitoring of patient status and therapy delivered to patients suffering from conditions such as heart failure in a manner the reduces the rate of heart failure patient readmission to a hospital. Some of the embodiments described herein are based on alert generation in conjunction with heart failure monitoring, although the invention is applicable alert generation for any type of condition which causes an acute deleterious change in a physiologic parameter indicating hospital readmission may be necessary, including exemplary conditions producing tachypnea.

Respiration rate has been shown to be predictive of mortality in a heart failure patient population. Dyspnea (primarily caused by tachypnea) is among the primary reasons why many heart failure patients return to the hospital during a HF decompensation episode. In the chronic, non-decompensated state, heart failure patients have elevated respiration rates. These rates become even more highly elevated in association with decompensation even at rest. Thus, for many patients, respiration rate provides a valuable indication or prediction of impending acute decompensation of HF. Information developed from respiratory rate data in accordance with embodiments of the present invention provides for enhanced monitoring and therapy management of heart failure patients that allows for early intervention, particularly during a time following hospital discharge and for purposes of reducing heart failure patient readmission rates.

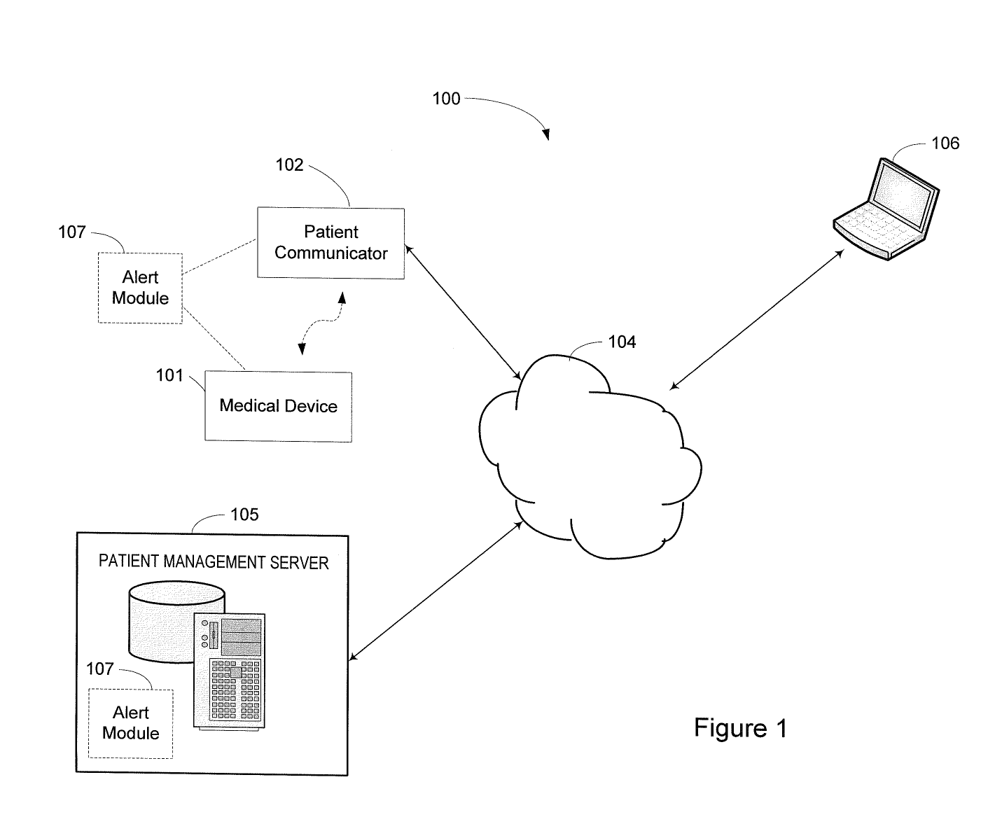

FIG. 1 is a block diagram of a system for implementing various processes of hospital readmission alert generation for heart failure patients in accordance with embodiments of the invention. The diagram of FIG. 1 illustrates a system 100 that may be configured to implement the processes described herein. According to various embodiments, processes described herein may be implemented by all or a subset of the elements shown in FIG. 1. In some embodiments, a medical device 101 incorporates or otherwise is coupled to an alert module 107 that operate cooperatively to implement the processes described herein. In other embodiments, the medical device 101 incorporates or otherwise is coupled to an alert module 107 that operate cooperatively with a local or remote processing device or system (e.g., patient communicator 102, personal computer (PC) 106, and/or patient management server 105) to implement the processes described herein. It will be understood that various embodiments of the present invention can be implemented using all or selected elements (and other or alternative elements described herein) shown in FIG. 1. It will be further understood that some embodiments include at least one implantable element, while other embodiments include only external elements.

The following discussion of FIG. 1 presents an embodiment wherein information acquired by a medical device 101 and/or patient communicator 102 is transmitted to an alert module 107 of a patient management server 105. The alert module 107 is generally described as having the functionality to assess changes in patient status and/or therapy effectiveness based on comparison of parameters to alert criteria. It will be appreciated that the alert module 107 need not be located in the patient management server 105, but may alternatively be located in the medical device 101, the patient communicator 102, or the PC 106. It will be further appreciated that components of the alert module 107 may be incorporated across multiple devices 101, 102, 105, 106 (or other devices).

The patient is instrumented with an implanted, patient-worn, or patient-carried medical device 101 that communicates with a patient communicator 102. The patient communicator 102 may be a portable device, a bed-side device, a programmer, a PC 106 equipped with appropriate communication software and hardware, or other type of device configured to effect communications with the medical device 101 and the patient management server 105. For example, the medical device 101 may be a cardiac rhythm management (CRM) device or other type of implantable diagnostic and/or therapeutic device (e.g., respiration monitor) that is implanted in the patient.

The medical device 101 and/or the patient communicator 102 are equipped with sensors configured to monitor various physiologic parameters. Suitable physiologic parameters include respiration, respiration data, blood pressure data, heart sounds data, heart rate variability data, lead impedance data, electrogram data, electrocardiogram data, electrical activation sequence data, cardiac vector axis data, QRS axis data, mechanical sensor data, mechanical contractile sequence data, vascular tone data, neuro-hormonal data, hemodynamic data, intrathoracic impedance data, transthoracic impedance data, intracardiac impedance data, heart sounds data, physiological response to activity data, pulmonary arterial pressure data, central venous pressure data, electro-mechanical delay data, patient weight data, and patient posture data. The medical device 101 stores information about the physiologic parameters it senses and, at periodic intervals, on command, or on an event-driven basis, the medical device 101 downloads the stored physiological information to the communicator 102. Other forms of data, such as patient symptom questionnaire data and externally measured blood pressure, may be communicated to at least one of the patient communicator 102 and the patient management server 105 (e.g., directly for weight and blood pressure sensors and indirectly via a PC for inputting patient symptom questionnaire data).

The patient communicator 102 is communicatively coupled to the patient management server 105 via a network 104, such as the Internet. The patient communicator 102 transmits the information acquired from the medical device 101 to the patient management server 105 for additional analysis. In addition to transmitting the information acquired by the medical device 101, the patient communicator 102 may also send to the patient management server 105 data that the patient communicator 102 has acquired through its own physiological sensors or other sensor with which the patient communicator 102 communicates, or via patient input.

At the patient management server 105, the data is stored and analysis of the patient condition and/or therapy effectiveness is performed by the alert module 107. As a part of this analysis, the physiologic parameters are calculated and are compared to alert criteria. As previously mentioned, in alternate embodiments, computing and comparison of the physiologic parameters to the alert criteria may be performed by the patient communicator 102 or by the implantable device 101. The physiologic parameter information may be trended and may be made available for remote access by a physician through a network-connected computer 106. When the parameters meet alert criteria, an alert signal may be generated to notify the physician or other action may be taken based on the alert signal.

Methodologies described herein advantageously provide physicians with a quantified metric that can be used to monitor a patient's changing heart failure status and/or evaluate the effectiveness of therapy (e.g., drug or cardiac stimulation therapy) delivered to the patient. In particular, methodologies described herein provide for determining worsening of a patient's heart failure status following hospitalization for a clinical HF event and generating a readmission alert if it appears that the heart failure patient will require readmission because of the worsening HF status of the patient. Methodologies of the present invention advantageously allow physicians to intervene early after discharge of a heart failure patient from the hospital when indications are detected that hospital readmission may be required, thereby allowing for interventional treatment to commence and avoidance of hospital readmission.

According to embodiments of the present invention that employ detection and analysis of one or more patient respiration parameters, methodologies for developing respiration data preferably involve measuring values of a respiration characteristic, which may be respiration rate, but could also be breath interval, tidal volume, and/or other respiration characteristics. The respiration characteristic measurements may be made for one or more breath cycles during a plurality of time apertures, which may or may not be overlapping in time. An estimated respiration characteristic, e.g., estimated rate, breath interval, tidal volume, etc, may be determined from the set of measured characteristic values for a particular aperture to summarize the measurements for the particular aperture. In one implementation, the median value of the respiration characteristic measurements made during an aperture is used to estimate the respiration characteristic of the aperture.

Other statistical estimates of respiration parameters (e.g., mean respiration rate) or non-statistical estimates (e.g., based on measured morphological characteristics of the respiration signal) may alternatively be used. The estimated respiration characteristics of a plurality of apertures may be used to develop a respiration trend, or may be used to derive a respiration metric that spans a period of time, such as a daily value. An estimated respiration characteristic may be estimated based on the measured respiration characteristic values of an individual aperture. Respiration metrics, such as daily respiration metrics, may be determined based on the estimated respiration characteristics of a plurality of apertures.

One implementation involves the use of a median estimator to determine daily respiration rate metrics, such as maximum respiration rate (MaxRR) and median respiration rate (MedRR) over a period of time. A daily minimum respiration rate (MinRR) may also be determined. Embodiments of the invention are directed to use of both MaxRR and MedRR to provide for enhanced monitoring of a patient's tachypnea status. In particular, it has been found through clinical investigation that the combined use of MaxRR and MedRR provides for superior detection of change in a patient's tachypnea status when compared to individual use of MaxRR or MedRR. It has been further found through clinical investigation that patient activity level can be used to reduce the occurrence rate of false positives. It has also been found through clinical investigation that the MinRR metric can be used to reduce the occurrence rate of false positives.

The daily maximum respiration rate may be best interpreted by considering it in association with the patient's daily activity. In a healthy, active patient, the maximum respiration rate will be significantly higher than the minimum value, and will vary considerably from day to day, reflecting the variability in the patient's activities. If elevated maximum respiration rates are associated with periods of very limited activity, the patient may be experiencing exertional dyspnea even at low levels of exertion (for example, simply walking around the house), which may indicate worsening patient status. A person whose activities are severely limited by health conditions may show less of a spread from minimum to maximum and/or less day-to-day variability in maximum respiration rate, due to limited, consistent daily activity patterns.

The median respiration rate is representative of the predominant respiration rate for a given time period. The daily median is relatively insensitive to transiently elevated respiration rates during periods of high activity, and also relatively insensitive to the lowest respiration rates typically occurring during deep sleep.

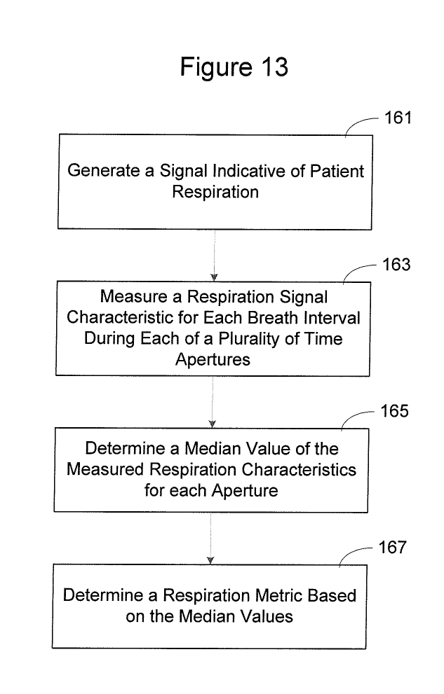

In various embodiments, a patient's daily maximum respiration rate and daily median respiration rate are determined. The patient's daily minimum respiration rate may optionally be determined. In these embodiments, the patient's respiration rate is measured for each breath cycle in a plurality of time apertures that cover about a 24 hour period. The median respiration rate is estimated for each time aperture. The daily maximum respiration rate is determined as the maximum median respiration rate of the time apertures spanning the 24 hour period. The daily median respiration rate may be determined as the median of the median respiration rates estimated for all of the time apertures that span the 24 hour period. In another implementation, the daily median respiration rate may be determined as the median value of all the respiration rate values measured over the 24 hour period. The daily minimum respiration rate is determined as the minimum median respiration rate of the time apertures spanning the 24 hour period. Additional details for deriving these respiration metrics are described hereinbelow beginning with FIG. 13.

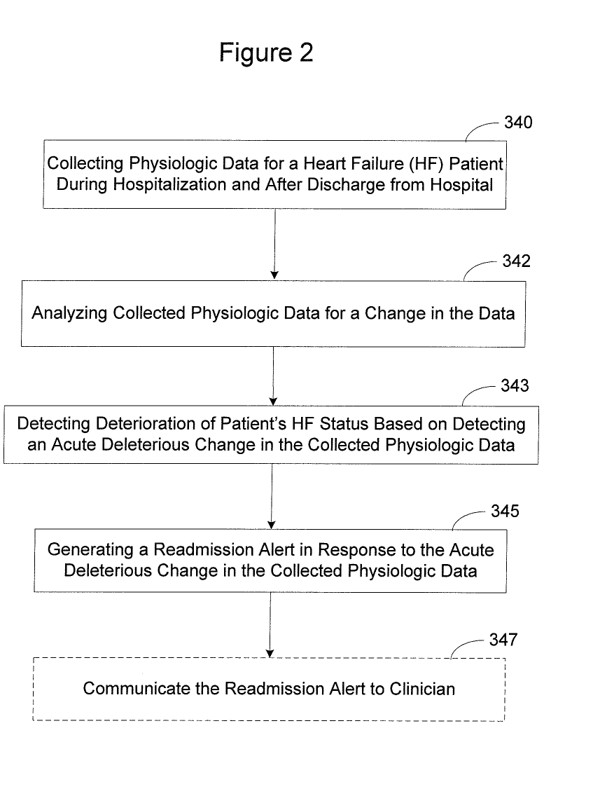

FIG. 2 is a flow chart illustrating various processes for determining worsening of a patient's heart failure status following hospitalization for a clinical HF event and generating a readmission alert if it appears that the heart failure patient will require readmission because of the worsening HF status of the patient in accordance with embodiments of the present invention. The methodology illustrated in FIG. 2 involves collecting 340 physiologic data for a heart failure patient during hospitalization and after discharge of the heart failure patient from a hospital, and analyzing 342 the collected physiologic data.

The methodology illustrated in FIG. 2 further involves detecting 343 deterioration of the patient's heart failure status based at least in part on detecting an acute deleterious change in the collected physiologic data indicative of an abnormality in one or more physiologic parameters represented by the physiologic data. A readmission alert is generated 345 in response to detecting the acute deleterious change in the collected physiologic data. The readmission alert preferably indicates that the deterioration in the patient's heart failure status may require readmission of the patient to the hospital. In various embodiments, the readmission alert is preferably communicated 347 to a clinician or patient advocate.

The methodology illustrated in FIG. 2 may involve receiving a discharge notification associated with a time of heart failure patient discharge from the hospital, and the physiologic data used for readmission alert generation are collected during hospitalization and after the time associated with patient discharge. Physiologic data collected during patient hospitalization is preferably used to define a baseline for the physiologic data and a threshold. Detecting an acute deleterious change in the collected physiologic data preferably involves comparison of one or more respiration metrics measured after patient discharge from the hospital to one or more thresholds developed during patient hospitalization.

For example, a threshold may be developed using physiologic data collected during a specified period when the heart failure patient is in the hospital, such as seven days before the day of discharge. In another embodiment, the baseline and the threshold can be established by analyzing the data during a period when the patient is stable or when there is no heart failure event. The acute deleterious change in the collected physiologic data may be detected within a window of about one day to about two months after discharge of the heart failure patient from the hospital. In other embodiments, the acute deleterious change in the collected physiologic data may be detected within a window of about one day to about one month after discharge of the heart failure patient from the hospital.

Detecting deterioration of the patient's heart failure status may involve comparing the change in the collected physiologic data to a threshold comprising at least one of a predetermined change in the collected physiologic data, a predetermined rate of change in the collected physiologic data, and a predetermined variance in the collected physiologic data. The physiologic data collected for processing by a readmission alert algorithm of the present invention may comprises at least one of respiration data, respiration metrics, blood pressure data, intra-abdominal pressure sensor data, heart sounds data, heart rate variability data, lead impedance data, electrogram data, electrocardiogram data, electrical activation sequence data, cardiac vector axis data, QRS axis data, mechanical sensor data, mechanical contractile sequence data, vascular tone data, neuro-hormonal data, hemodynamic data, intrathoracic impedance data, transthoracic impedance data, intracardiac impedance data, heart sounds data, physiological response to activity data, pulmonary arterial pressure data, central venous pressure data, electro-mechanical delay data, patient weight data, patient posture data, and patient symptom questionnaire data.

In some embodiments, a readmission alert generation algorithm of the present invention is implemented by an implantable medical device. In other embodiments, a readmission alert generation algorithm of the present invention is implemented at least in part by an implantable medical device. In various embodiments, a readmission alert generation algorithm of the present invention is implemented by an external medical device. In further embodiments, a readmission alert generation algorithm of the present invention is implemented in part by an implantable medical device and in part by an external device or system.

In various implementations, the external device or system is a local patient management system, such as a PC or other electronic device that communicates with the patient medical device (implantable or external). In other implementations, the external device or system is a local communication interface (e.g., mobile patient communicator, PC, or bed-side monitor) that effects communication with a remote patient management server. Accordingly, various embodiments are contemplated in which various data collection, processing/analysis, and alert generation functions are performed by one or more implantable medical devices, external medical devices, local communication/processing devices, remote communication/processing devices, and any combination of these modes of implementation.

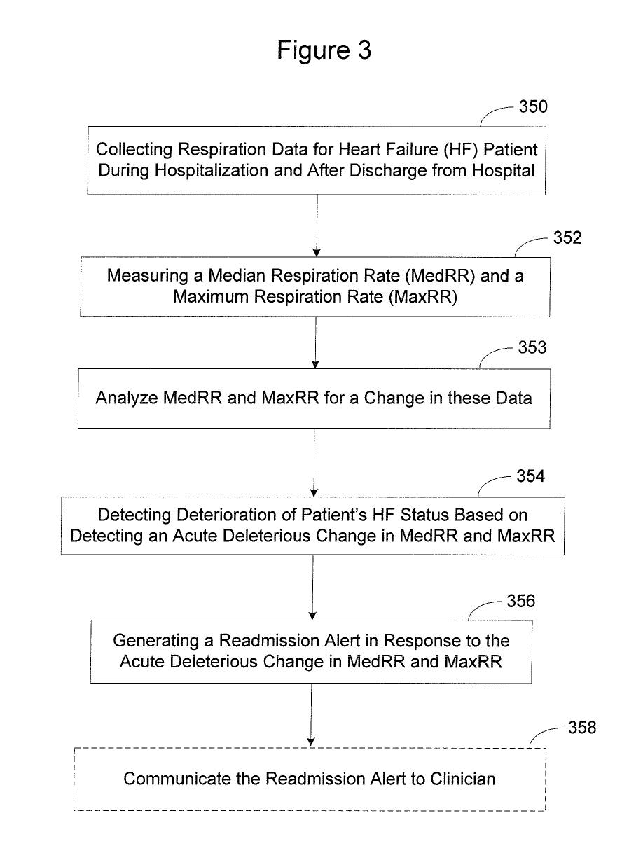

FIG. 3 is a flow chart illustrating various processes for determining worsening of a patient's heart failure status following hospitalization for a clinical HF event and generating a readmission alert if it appears that the heart failure patient will require readmission because of the worsening HF status of the patient in accordance with embodiments of the present invention. The methodology illustrated in FIG. 3 involves collecting 350 respiration data for a heart failure patient during hospitalization and after discharge of the heart failure patient from a hospital, and measuring 352 a median respiration rate (MedRR) and a maximum respiration rate (MaxRR) using the collected respiration data.

The methodology illustrated in FIG. 3 further involves analyzing 353 the MedRR and MaxRR for a change in the MedRR and MaxRR and detecting 354 deterioration of the patient's heart failure status based at least in part on detecting an acute deleterious change in the MedRR and MaxRR indicative of an abnormality in MedRR and MaxRR. A readmission alert is generated 356 in response to detecting the acute deleterious change in the MedRR and MaxRR. The readmission alert preferably indicates that the deterioration in the patient's heart failure status may require readmission of the patient to the hospital. In various embodiments, the readmission alert is preferably communicated 358 to a clinician or patient advocate.

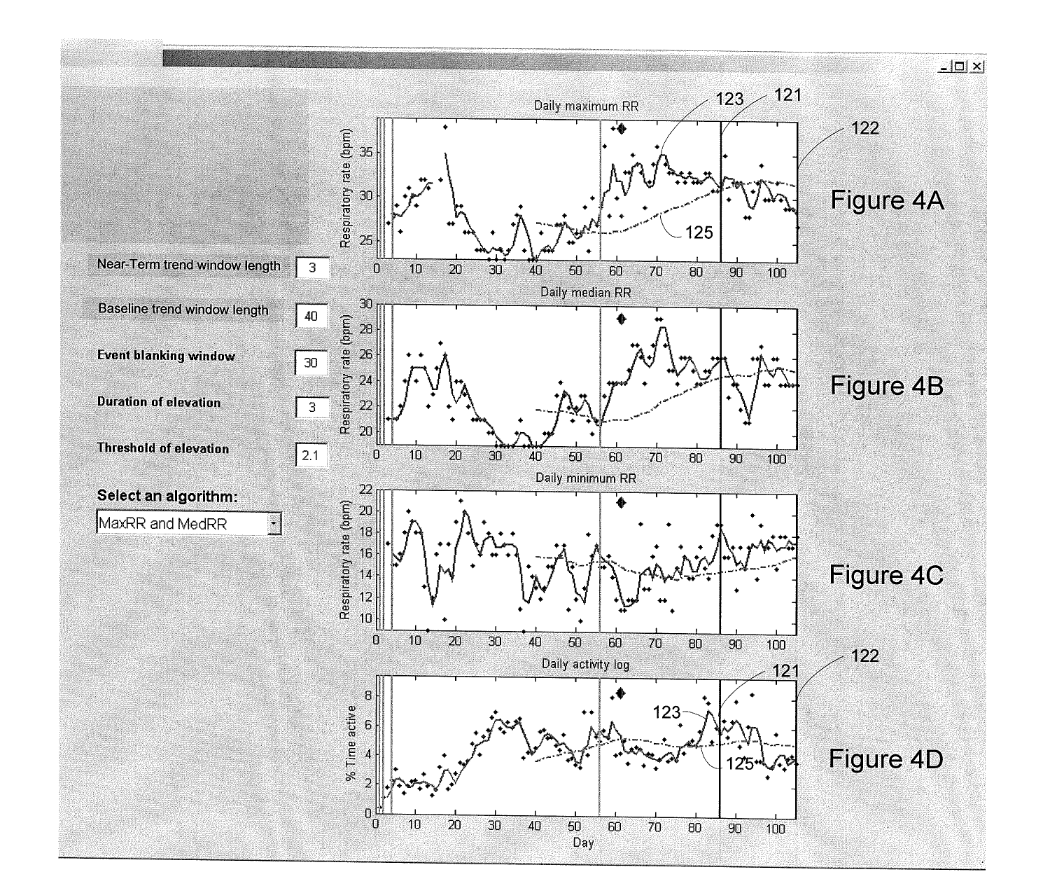

FIG. 4 shows representative plots of different respiration rate metrics and activity data associated with methodologies for detecting early onset of worsening heart failure in accordance with embodiments of the present invention. FIGS. 4A, 4B, and 4C are plots of near-term averages 123 and baseline averages 125 for MaxRR, MedRR, and MinRR, respectively, developed from respiration data for a particular patient. FIG. 4D is a plot of patient activity data (e.g., accelerometer data). Two HF event alerts are shown as lines 121 and 122 for this representative patient.

In the context of FIG. 4, an HF event is declared whenever the patient has signs and/or symptoms consistent with congestive HF and (a) the patient receives unscheduled intravenous therapy (e.g., intravenous (IV) diuretics, IV inotropes, IV vasoactive drugs), oral thiazide, or ultrafiltration therapy that does not involve formal in-patient hospital admission, regardless of the setting (i.e. in an emergency room setting, in the physician's office, etc.) or (b) one of the patient's reasons for admission to the hospital was HF and the patient received an augmented heart failure regimen with oral or intravenous medications or ultrafiltration therapy (formal hospital admission is defined as admission to the hospital that includes a calendar date change).

Arranged vertically along the left panel of FIG. 4 are parameters that are used by the HF event detection algorithm that processes the data of FIGS. 4A-4D. These parameters include the near-term window length (e.g., 3 days), baseline window length (e.g., 40 days), event blanking window (e.g., 30 days), duration of elevation (e.g., 3 days), and threshold of elevation (e.g., 2.1). The near-term and baseline windows have been previously discussed. The event blanking window represents a period of time following generation of an alert for which generation (or communication) of a subsequent alert is not permitted. The purpose of the event blanking window is to prevent repeated alerting of the same patient condition that generated an initial alert.

It is noted that the baseline average respiration metrics are updated during the event blanking window. There are conditions, however, when updating of the baseline average respiration metrics may not be permitted or is modified. For example, it may be desirable not to permit updating of the baseline average respiration metrics once it is determined that the patient is in a disease condition. In this case, the baseline average respiration metrics are not updated until the disease condition is resolved. In another scenario, it may be desirable to permit updating of the baseline average respiration metrics but in a modified form. For example, respiration rate data within the baseline window may be weighted in a manner that de-emphasizes data collected during or surrounding an HF event.

The duration of elevation parameter and threshold of elevation operate cooperatively. The threshold of elevation was discussed previously, and, in general terms, modifies the relationship between detection sensitivity and rate of false positives. The duration of elevation represents the amount of time the near-term average respiration rate metric must exceed its associated baseline average respiration rate metric before an alert condition is considered verified.

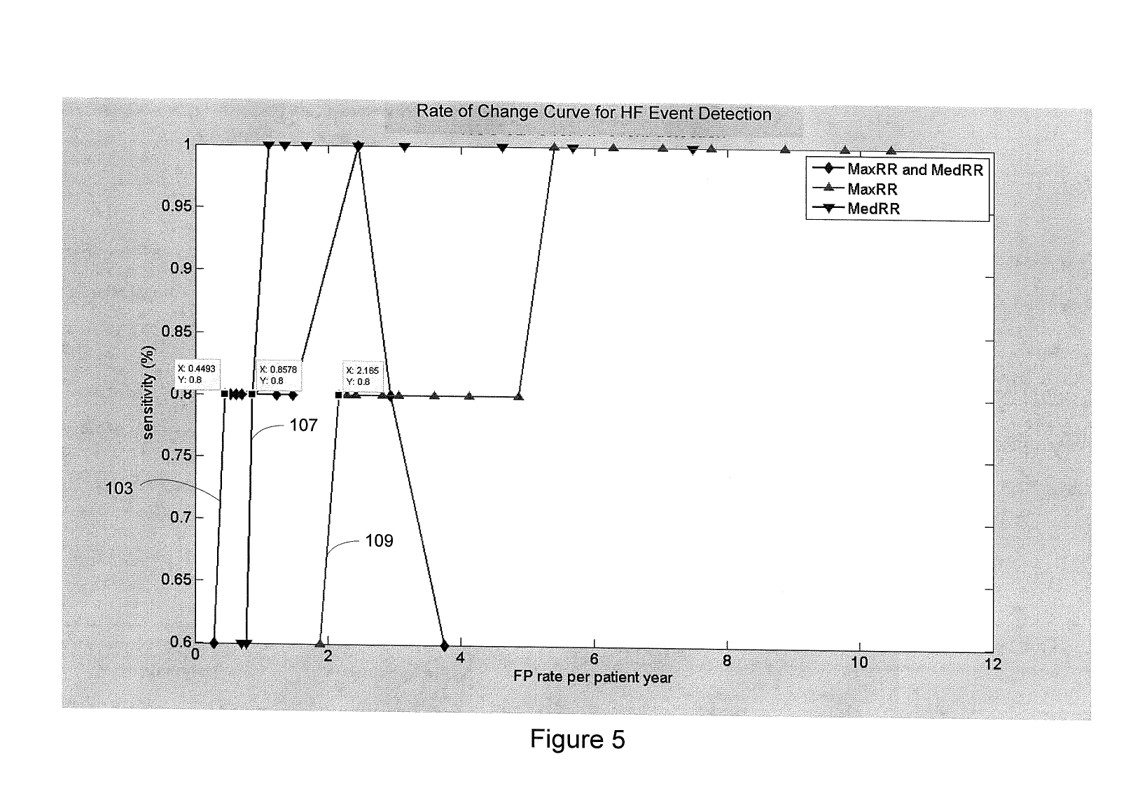

FIG. 5 graphically illustrates an improvement in detecting early onset of worsening heart failure status of patients when using both MaxRR and MedRR metrics in accordance with embodiments of the invention. FIG. 5 is a plot of the rate of false positives per patient year (x-axis) as a function of detection sensitivity (y-axis). Plots for MedRR 107, MaxRR 109, and combined MaxRR and MedRR 103 are shown in FIG. 5.

At an optimized detection sensitivity of 80%, the rates of false positives for the three HF event detection methodologies shown in FIG. 5 are as follows: HF event detection using MedRR 107 resulted in a false positive rate per patient year of approximately 0.86; HF event detection using MaxRR 109 resulted in a false positive rate per patient year of approximately 2.17; and HF event detection using combined MaxRR and MedRR 103 resulted in a false positive rate per patient year of approximately 0.45. It is noted that HF event detection using combined MaxRR and MedRR 103 at a detection sensitivity of 60% resulted in a false positive rate per patient year of approximately 0.29.

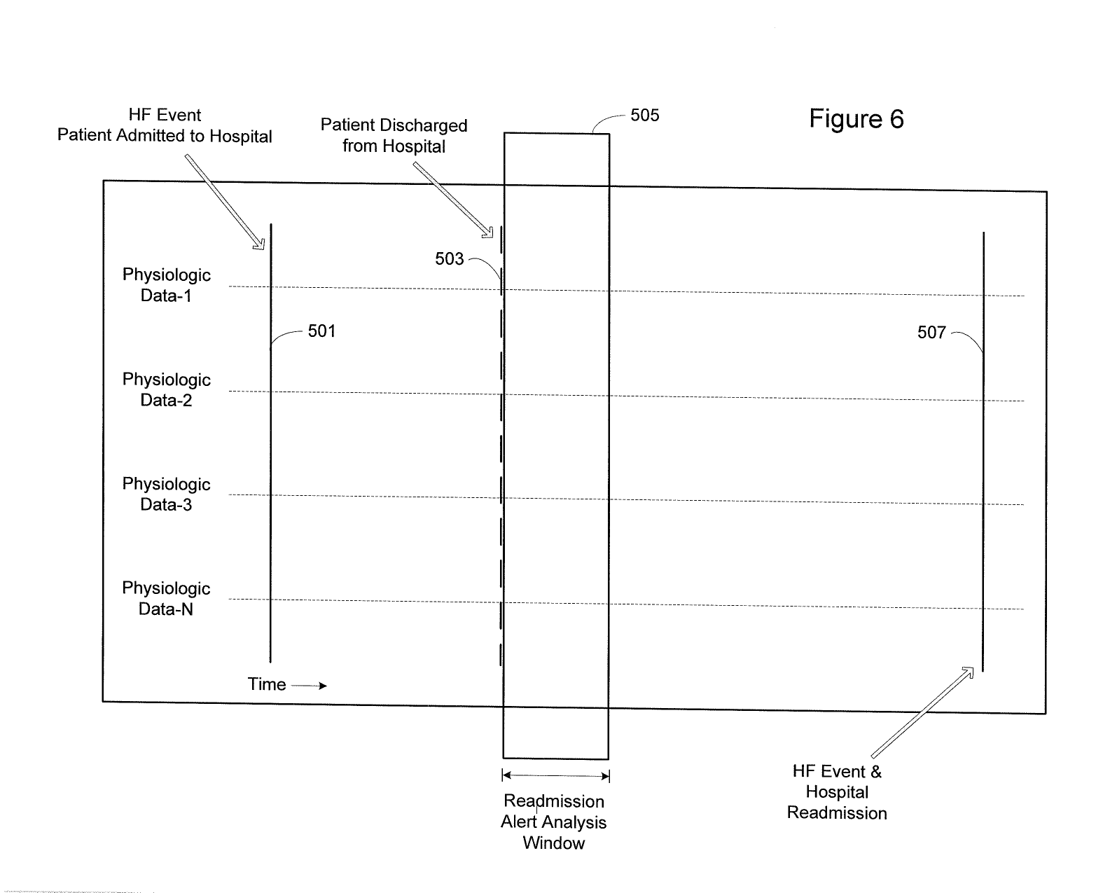

FIG. 6 is a chart that illustrates relationships between various events in connection with detecting early onset of worsening heart failure and generation of a hospital readmission alert in accordance with embodiments of the present invention. FIG. 6 shows locations for representative plots (no data shown for purposes of simplicity) of different physiologic data (Physiologic Data-1 through Physiologic Data-N) for a particular heart failure patient as a function of time. In this illustrative scenario, it is assumed that the heart failure patient is experiencing or has recently experienced an HF event requiring transport to a hospital or clinic equipped to handle heart failure patients. At time 501, it is assumed that the heart failure patient has suffered a clinical HF event and has been admitted to the hospital. During a time period defined between times 501 and 503, the heart failure patient is subject to treatment during his or her stay at the hospital. At time 503, it is assumed that the heart failure patient's status has improved to a point where hospitalization is no longer required or warranted. At time 503, the patient is discharged from the hospital.

During at least a portion of the patient's stay in the hospital (a period(s) between times 501 and 503), physiologic data is collected for purposes of developing a baseline(s) and threshold(s) that are used by a hospital readmission alert algorithm of the present invention. For example, physiologic data may be collected during the last seven days prior to patient discharge for purposes of developing baseline and threshold values for use by the hospital readmission alert algorithm. In another embodiment, the baseline and the threshold can be established by analyzing the data during a period when the patient is stable or when there is no heart failure event.

At or near the time 503 the patient has been discharged, the hospital readmission alert algorithm initiates collection and analysis of post-discharge physiologic data acquired for the patient. In the illustrative example of FIG. 6, physiologic data shown as Physiologic Data-1 through Physiologic Data-N are collected for the patient during a readmission alert analysis window 505. In general terms, the readmission alert analysis window 505 defines a duration of time following patient discharge from the hospital for collection and analysis of the discharged patient's physiologic data that is sufficiently long to detect early onset of worsening heart failure post-discharge and generate a hospital readmission alert in accordance with embodiments of the present invention.

Analysis of patient data within the readmission alert analysis window 505 may occur on a periodic or continuous basis. This analysis or analyses may be performed by the medical device(s) that is/are acquiring the physiologic data (e.g., implantable or external medical device), a separate device or system (local or remote) that is communicatively coupled to the physiologic data acquisition device(s), or a combination of these and/or other devices/systems.

If, during the readmission alert analysis window 505, degradation of the patient's HF status is detected such that hospitalization may soon be required if physician intervention does not occur, a hospital readmission alert is generated and communicated to the patient's physician, healthcare advocate (e.g., such as by way of a server-based patient management system), and/or to the patient. In response to the hospital readmission alert, the patient's physician preferably intervenes and provides appropriate treatment to the patient, typically by requesting the patient to call or visit the physician or clinic (e.g., such as for addition or adjustment of a drug regimen and/or medical device therapy). Such intervention and treatment advantageously occurs after generation of the hospital readmission alert so that readmission of the patient to the hospital is avoided. Time 507 in FIG. 6 represents occurrence of a subsequent clinical HF event that would have required readmission of the patient to the hospital in the absence of detecting early onset of worsening heart failure and generation of a hospital readmission alert in accordance with embodiments of the present invention.

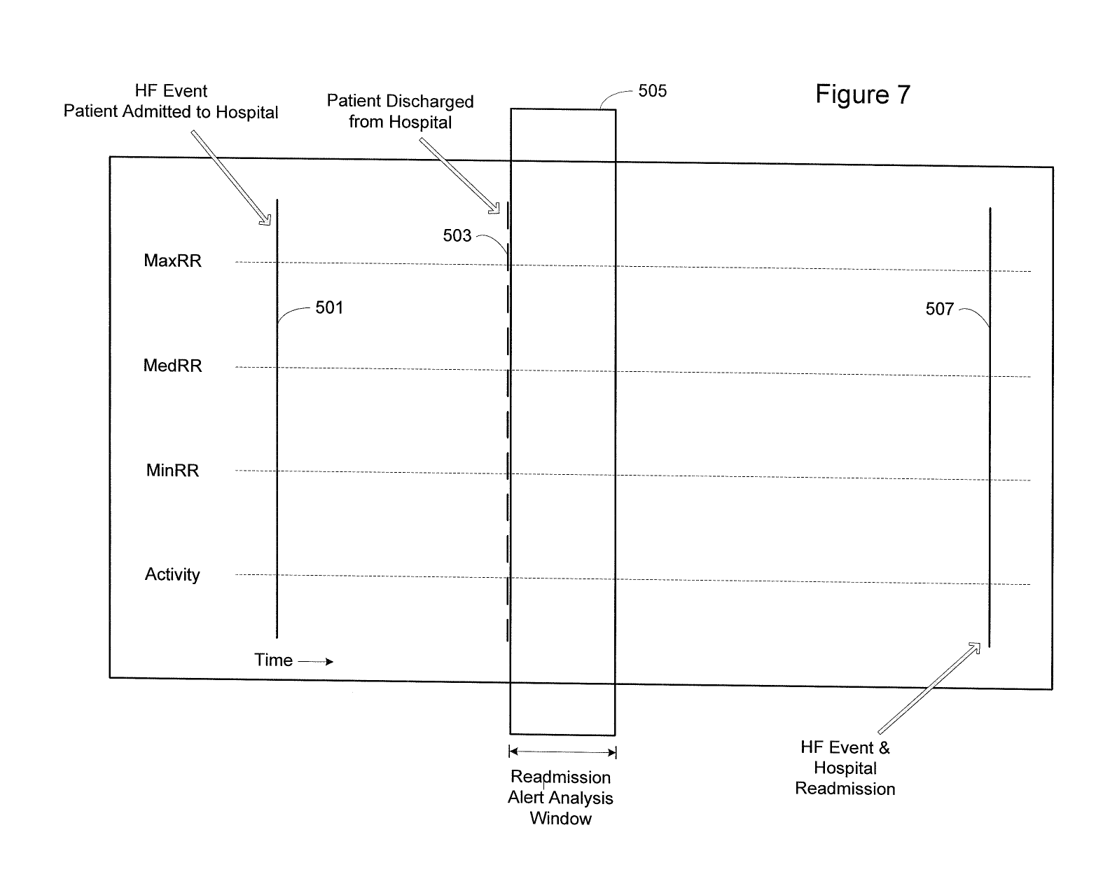

FIG. 7 is a chart that illustrates relationships between various events in connection with detecting early onset of worsening heart failure and generation of a hospital readmission alert in accordance with other embodiments of the present invention. FIG. 7 shows locations for representative plots (no data shown for purposes of simplicity) of different respiration and patient activity data for a particular heart failure patient as a function of time. In the illustrative scenario shown in FIG. 7, MaxRR, MedRR, and MinRR metrics are measured, as is patient activity. In some embodiments, only MaxRR and MedRR are measured and analyzed for purposes of detecting early onset of worsening heart failure and generating a hospital readmission alert. In other embodiments, one or both of MinRR and patient activity are also measured and analyzed for purposes of detecting early onset of worsening heart failure and generating a hospital readmission alert.

As in the case of the scenario illustrated in FIG. 6, it is assumed in FIG. 7 that the heart failure patient is experiencing or has recently experienced an HF event requiring transport to a hospital or clinic equipped to handle heart failure patients. At time 501, it is assumed that the heart failure patient has suffered a clinical HF event and has been admitted to the hospital. During a time period defined between times 501 and 503, the heart failure patient is subject to treatment during his or her stay at the hospital. At time 503, it is assumed that the heart failure patient's status has improved to a point where hospitalization is no longer required or warranted, and the patient is discharged from the hospital at time 503.

During at least a portion of the patient's stay in the hospital (a period(s) between times 501 and 503), respiration data is collected for purposes of developing baselines for one or more of the respiration metrics, MaxRR, MedRR, and MinRR, and optionally patient activity, and their respective threshold(s) that are used by a hospital readmission alert algorithm of the present invention. For example, respiration data may be collected during the last seven days prior to patient discharge for purposes of developing baseline and threshold values for those respiration and patient activity metrics used by the hospital readmission alert algorithm. In another embodiment, the baseline and the threshold can be established by analyzing the data during a period when the patient is stable or when there is no heart failure event.

At or near the time 503 the patient has been discharged, the hospital readmission alert algorithm initiates collection and analysis of post-discharge patient respiration data and optional activity data. Using the collected data, at least MaxRR and MedRR, and optionally one or both of MinRR and patient activity data, are measured and analyzed during the readmission alert analysis window 505. This analysis or analyses may be performed by a medical device(s) that is/are acquiring the respiration and optional activity data (e.g., implantable or external medical device), a separate device or system (local or remote) that is communicatively coupled to the respiration/activity data acquisition device(s), or a combination of these and/or other devices/systems.

If, during the readmission alert analysis window 505, degradation of the patient's respiration metric(s) indicative of worsening HF status is detected such that hospitalization may soon be required if physician intervention does not occur, a hospital readmission alert is generated and communicated to the patient's physician or healthcare advocate. In response to the hospital readmission alert, the patient's physician preferably intervenes and provides appropriate treatment to the patient, so that hospital readmission of the patient is avoided.

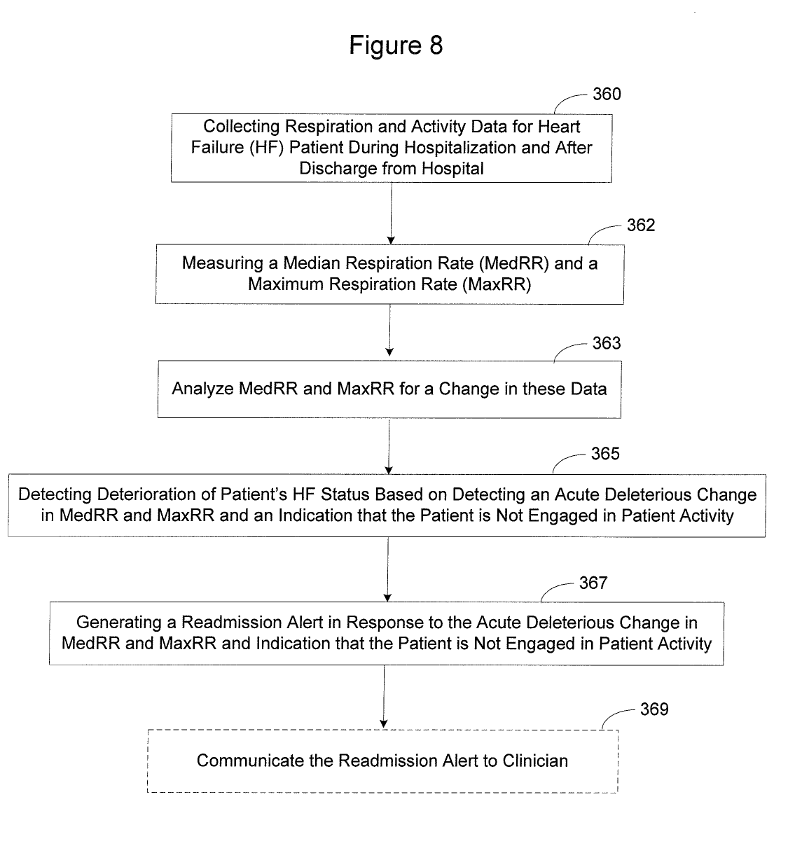

FIG. 8 is a flow chart illustrating various processes for determining worsening of a patient's heart failure status following hospitalization for a clinical HF event and generating a readmission alert if it is likely that the heart failure patient will require readmission because of worsening heart failure in accordance with embodiments of the present invention. The methodology illustrated in FIG. 8 involves collecting 360 respiration data and activity data for a heart failure patient during hospitalization and a time after discharge from the hospital, and measuring 362 a median respiration rate (MedRR) and a maximum respiration rate (MaxRR) using the collected respiration data. During at least a portion of the patient's stay in the hospital, respiration data is collected for purposes of developing baselines for the MedRR, MaxRR, and patient activity, and their respective thresholds that are used by a hospital readmission alert algorithm of the present invention.

The methodology illustrated in FIG. 8 further involves analyzing 363 the MedRR and MaxRR for a change in these data relative to their respective baselines and thresholds, and detecting 365 deterioration of the patient's heart failure status based at least in part on detecting an acute deleterious change in the MedRR and MaxRR indicative of an abnormality in MedRR and MaxRR and an indication that the patient is not engaged in patient activity. A readmission alert is generated 367 in response to detecting the acute deleterious change in the MedRR and MaxRR and an indication that the patient is not engaged in patient activity. The readmission alert preferably indicates that the deterioration in the patient's heart failure status may require readmission of the patient to the hospital. In various embodiments, the readmission alert is preferably communicated 369 to a clinician or patient advocate.

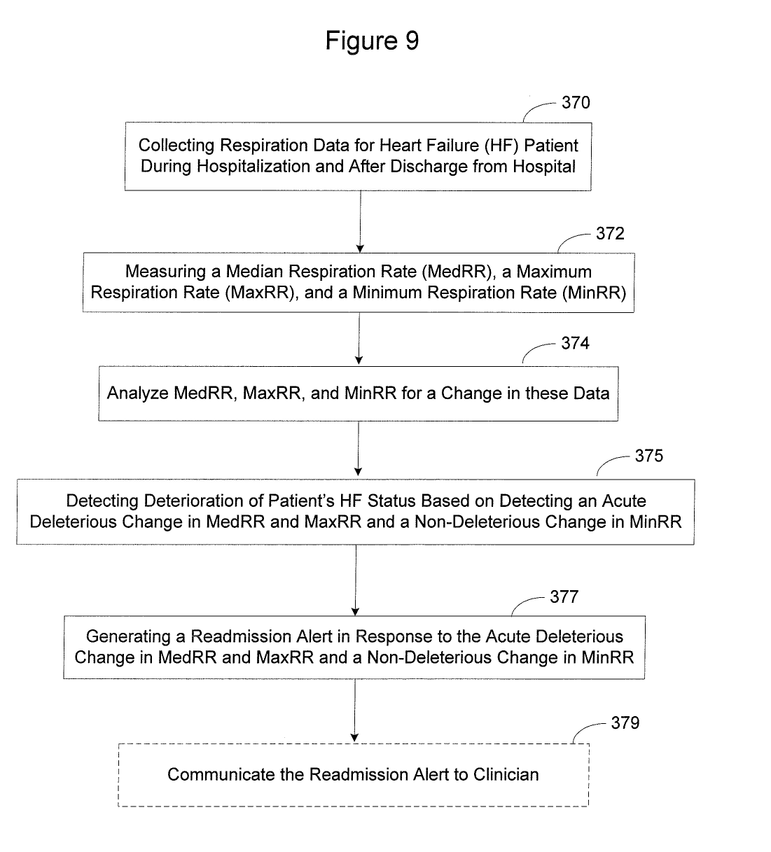

FIG. 9 illustrates various processes for determining worsening of a patient's heart failure status following hospitalization for a clinical HF event and generating a readmission alert if it is likely that the heart failure patient will require readmission because of worsening heart failure in accordance with other embodiments of the present invention. The methodology illustrated in FIG. 9 involves collecting 370 respiration data for a heart failure patient during hospitalization and a time after discharge from the hospital, and measuring 372 a median respiration rate (MedRR), a maximum respiration rate (MaxRR), and a minimum respiration rate (MinRR) using the collected respiration data. During at least a portion of the patient's stay in the hospital, respiration data is collected for purposes of developing baselines for the MedRR, MaxRR, and MinRR, and their respective thresholds that are used by a hospital readmission alert algorithm of the present invention.

The methodology illustrated in FIG. 9 further involves analyzing 374 the MedRR, MaxRR, and MinRR for a change in these data relative to their respective baselines and thresholds, and detecting 375 deterioration of the patient's heart failure status based at least in part on detecting an acute deleterious change in the MedRR and MaxRR indicative of an abnormality in MedRR and MaxRR, and a non-deleterious change in MinRR. A readmission alert is generated 377 in response to detecting the acute deleterious change in the MedRR and MaxRR and a non-deleterious change in MinRR. In various embodiments, the readmission alert is preferably communicated 379 to a clinician or patient advocate.

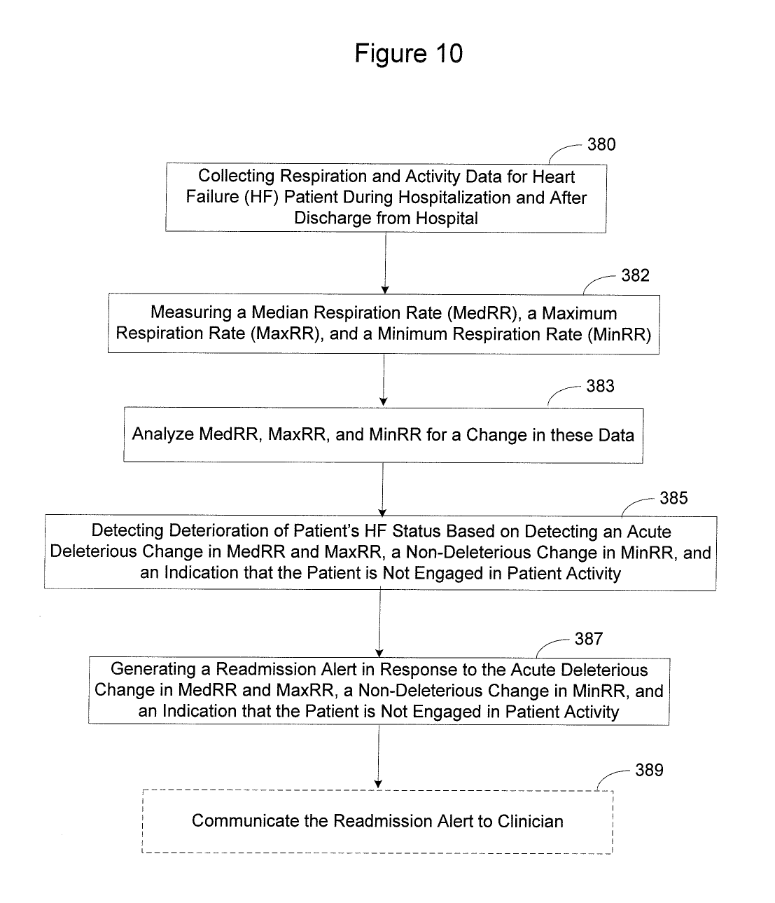

FIG. 10 illustrates various processes for determining worsening of a patient's heart failure status following hospitalization for a clinical HF event and generating a readmission alert if it is likely that the heart failure patient will require readmission because of worsening heart failure in accordance with further embodiments of the present invention. The methodology illustrated in FIG. 10 involves collecting 380 respiration data and activity data for a heart failure patient during hospitalization and a time after discharge from the hospital, and measuring 382 a median respiration rate (MedRR), a maximum respiration rate (MaxRR), and a minimum respiration rate (MinRR) using the collected respiration data. During at least a portion of the patient's stay in the hospital, respiration data is collected for purposes of developing baselines for the MedRR, MaxRR, MinRR, and patient activity, and their respective thresholds that are used by a hospital readmission alert algorithm of the present invention.

The methodology illustrated in FIG. 10 further involves analyzing 383 the MedRR, MaxRR, and MinRR for a change in these data relative to their respective baselines and thresholds. Deterioration of the patient's heart failure status is detected 385 based at least in part on detecting an acute deleterious change in the MedRR and MaxRR indicative of an abnormality in MedRR and MaxRR, a non-deleterious change in MinRR, and an indication that the patient is not engaged in patient activity. A readmission alert is generated 387 in response to detecting the acute deleterious change in the MedRR and MaxRR, a non-deleterious change in MinRR, and an indication that the patient is not engaged in patient activity. In various embodiments, the readmission alert is preferably communicated 389 to a clinician or patient advocate.

In some embodiments, detecting deterioration of the patient's heart failure status involves determining whether the acute deleterious change in the MedRR exceeds a first threshold, and determining whether the acute deleterious change in the MaxRR exceeds a second threshold. A readmission alert is preferably generated in response to the acute deleterious change in the MedRR and MaxRR exceeding the first and second thresholds, respectively.

The first threshold may be developed using the MedRR collected before the discharge of the heart failure patient from the hospital, and determining whether the acute deleterious change in the MedRR exceeds the first threshold is preferably based on a comparison of at least the MedRR collected after the discharge of the heart failure patient from the hospital relative to the MedRR collected before the discharge. The second threshold may be developed using the MaxRR collected before the discharge of the heart failure patient from the hospital, and determining whether the acute deleterious change in the MaxRR exceeds the second threshold is preferably based on a comparison of at least the MaxRR collected after the discharge of the heart failure patient from the hospital relative to the MaxRR collected before the discharge. In another embodiment, the baselines and the thresholds can be established by analyzing the data during a period when the patient is stable or when there is no heart failure event.

In some embodiments, the first and second thresholds may respectively be developed using respiration data collected during a period when the heart failure patient is in the hospital, such as seven days before the day of discharge. The acute deleterious change in the MedRR and MaxRR may be detected within a window of about one day to about two months after discharge of the heart failure patient from the hospital. In other embodiments, the acute deleterious change in the MedRR and MaxRR may be detected within a window of about one day to about one month after discharge of the heart failure patient from the hospital.

Implementation of a readmission alert algorithm of the present invention may involve collecting patient activity data for the patient, and generating a readmission alert in response to an acute deleterious change in the MedRR and MaxRR and the patient activity data indicating that the patient is not engaged in patient activity. Other implementations may involve measuring, using the respiration data, a minimum respiration rate (MinRR), analyzing the MinRR for a change in the MinRR, and generating an readmission alert in response to an acute deleterious change in the MedRR and MaxRR and a substantially non-elevated MinRR.

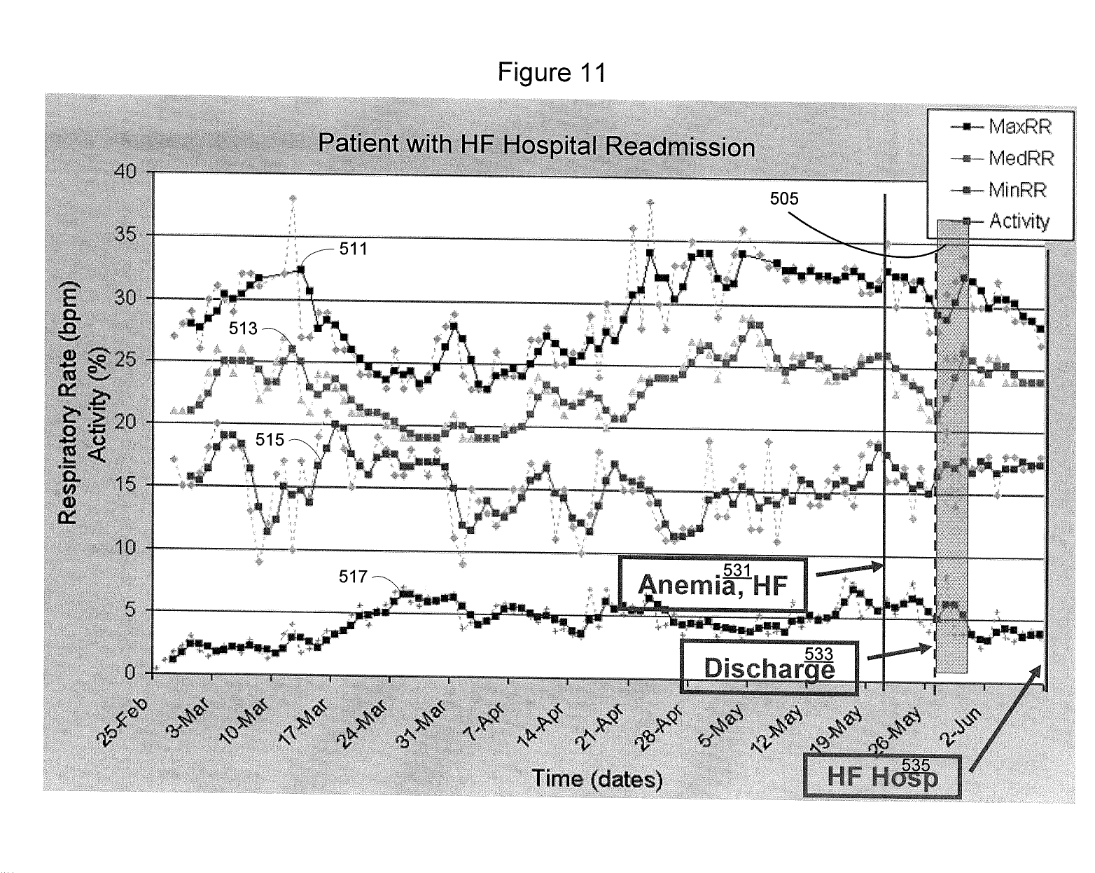

FIG. 11 is a representative plot of respiration data and activity data for a heart failure patient. The data shown in FIG. 11 includes MaxRR 511, MedRR 513, and MinRR 515 (in terms of breaths per minute), and patient activity 517 (in terms of percentage of time being active per day) plotted as a function of time over a period of approximately four months. FIG. 11 illustrates respiration and activity data for a heart failure patient that required readmission to the hospital relatively soon after HF treatment and discharge from the hospital.

Towards the later part of the plot, denoted by the first solid vertical line 531, the data indicates that the patient experienced a clinical event (anemia and an HF event) that required hospitalization. Improvement of the patient's condition can be seen in the data plotted between admission 531 and discharge 533 of the patient from the hospital.

After patient discharge 533 from the hospital, the data shows a fairly rapid or acute change in the patient's HF status that occurs within a window of time 505, followed by further worsening of the patient's HF status, ultimately leading to readmission 535 of the patient to the hospital for repeated HF treatment. The window 505 represents a readmission alert analysis window during which deterioration of the patient's heart failure status, as illustrated in FIG. 11, could have been detected based at least in part on detecting an acute deleterious change in the MedRR and MaxRR (and optionally MinRR) indicative of an abnormality in MedRR and MaxRR (and optionally MinRR) in accordance with embodiments of the invention. The activity data 517 indicates that the acute deleterious change in the MedRR and MaxRR (and optionally MinRR) was not due to the patient engaged in significant activity.

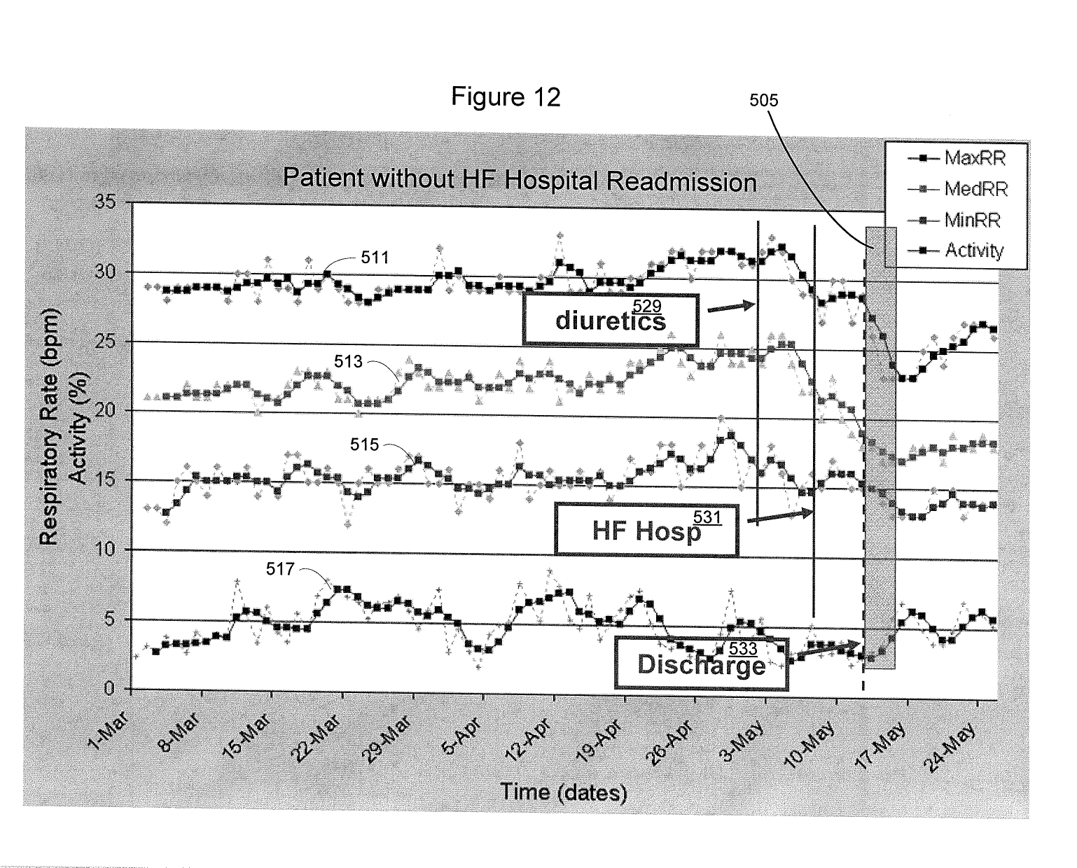

FIG. 12 is a representative plot of respiration data and activity data for a heart failure patient different from the patient discussed above with reference to FIG. 11. The data shown in FIG. 12 includes MaxRR 511, MedRR 513, and MinRR 515, and patient activity 517 plotted as a function of time over a period of approximately three months. FIG. 12 illustrates respiration and activity data for a heart failure patient that did not require readmission to the hospital after HF treatment and discharge from the hospital.

Towards the later part of the plot, the data indicates modest improvement of the patient's HF status resulting from the patient taking diuretics in-home, denoted by the solid line 529. This improvement, however, was not significant enough to prevent hospitalization, denoted by solid line 531. Also, a very gradual worsening of the patient's HF status can be seen in the data prior to line 529, indicated by a slow elevation in MaxRR and MedRR, which likely prompted the increase in diuretic dose at line 529. The data collected during hospitalization (denoted as the time between solid lines 531 and 533) shows further improvement in the patient's HF condition (mainly in MedRR) and right after discharge, indicated by line 533.

After discharge 533, respiration metrics and activity data were monitored within a readmission alert analysis window 505. The respiration metrics and activity data was analyzed within the readmission alert analysis window 505. Analysis of these data indicated that a readmission condition was not detected, and that readmission of the patient due to the patient's post-discharge HF status would be unnecessary or unwarranted.

FIG. 13 is a flow chart showing various processes of respiration rate trending that may be incorporated as part of a readmission alert algorithm in accordance with embodiments of the invention. As was previously discussed, methodologies used for developing respiration data may involve measuring values of a respiration characteristic, which may be respiration rate. The respiration characteristic measurements may be made for one or more breath cycles during a plurality of time apertures, which may or may not be overlapping in time. An estimated respiration characteristic may be determined from the set of measured characteristic values for a particular aperture to summarize the measurements for the particular aperture. In the embodiments shown in FIG. 13-16, the median value of the respiration characteristic measurements made during an aperture is used to estimate the respiration characteristic of the aperture.

The use of median estimators to derive respiration metrics is illustrated in the flowchart of FIG. 13. Patient respiration is sensed and a signal indicative of patient respiration is generated 161. The patient respiration signal may be generated, for example, by any of a variety of implantable or patient external sensors, such as an implantable transthoracic impedance sensor, external respiratory bands having piezoelectric or other sensor elements, a respiratory mask flow sensor, or other types of respiration sensors. A characteristic of the respiration signal, such as respiration rate per breath cycle, is measured 163 during each of a plurality of time apertures. The median value of the respiration characteristic measurements for each aperture is determined 165 and is used to estimate the respiration characteristic for the aperture. For example, if respiration rate is the measured characteristic, the median value of the respiration rates measured for each breath cycle during the aperture is determined. The median value is used to estimate the respiration rate of the aperture. One or more respiration metrics are determined 167 based on the estimated respiration characteristics (e.g., median values) of the apertures.

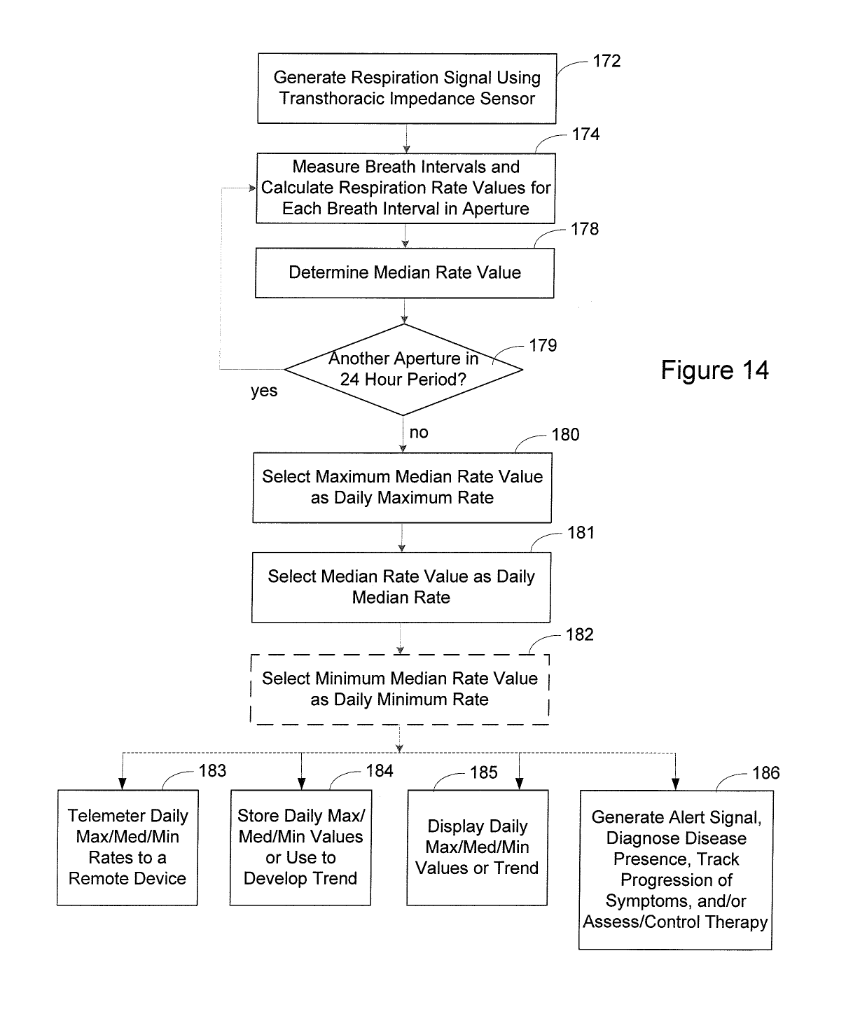

A method for generating and using daily respiration metrics is illustrated in FIG. 14. The process involves the use of an implantable transthoracic impedance sensor for determining a daily maximum and/or daily minimum respiration metric based on median estimators for the aperture respiration characteristics. In accordance with this embodiment, a respiration signal is generated 172 by a transthoracic impedance sensor implemented in conjunction with an implantable cardiac rhythm management (CRM) device or other implantable medical device. The transthoracic impedance sensor comprises intracardiac electrodes coupled to sensor drive/sense circuitry disposed within the CRM housing. The sensor drive circuitry delivers an electrical excitation signal, such as a strobed sequence of current pulses or other measurement stimuli, across the thorax via one set of the intracardiac electrodes.

In response to the drive current, a response voltage is sensed by the sense circuitry using another set of the intracardiac electrodes. The response voltage represents the transthoracic (i.e., across a portion of the chest or thorax) impedance. Transthoracic impedance sensing provides a voltage signal that tracks patient respiration and may be used to determine how fast and/or how deeply a patient is breathing. Additional aspects of transthoracic impedance sensing that may be utilized in conjunction with various embodiments of the present invention are described in commonly owned U.S. Pat. No. 6,076,015 which is incorporated herein by reference. In other embodiments, an external respiration sensor is used to detect patient respiration, it being understood that wholly external implementations of the present invention are contemplated.

A plurality of time apertures, covering about a 24 hour period, is superimposed relative to the generated respiration signal. The breath intervals occurring in each aperture are measured 174 and respiration rates for each breath cycle are calculated as the inverse of each measured breath interval. The median value of the measured respiration rates is computed and stored 178. Median values for each of the apertures are stored 179 throughout the 24 hour period. The maximum of the median values is selected 180 as the maximum daily respiration rate. The median of the median values is selected 181 as the median daily respiration rate. Optionally, the minimum of the median values is selected 182 as the minimum daily respiration rate.

The daily maximum and median rates (and optionally minimum rate) are stored or used 184 to develop trend data within the CRM device or remote device. The daily maximum and median rates (and optionally minimum rate) may optionally be telemetered 183 to a remote device. The daily maximum and median rates (and optionally minimum rate) or data developed from these metrics may optionally be displayed 185 on the device programmer screen or other user interface device as individual daily respiration metrics or as trended data.

The daily maximum and median rates (and optionally minimum rate) are preferably used 186 to generate an alert signal indicative of the patient's tachypnea status. The daily maximum and median rates (and optionally minimum rate) may be used 186 to generate an alert signal indicative of detection of early onset of worsening of the patient's heart failure status. The daily maximum and median rates (and optionally minimum rate) may be used 186 to generate an alert signal used for disease diagnosis, to track the progression of disease symptoms, and/or may to asses or control therapy. Although this example describes the use of daily metrics, other periodic metrics may also be determined, such as hourly metrics, weekly metrics, bi-weekly metrics, or monthly metrics. In addition, metrics other than maximum and minimum respiration rates may be determined, such as the daily, weekly, monthly, etc., median or mean respiration rates.

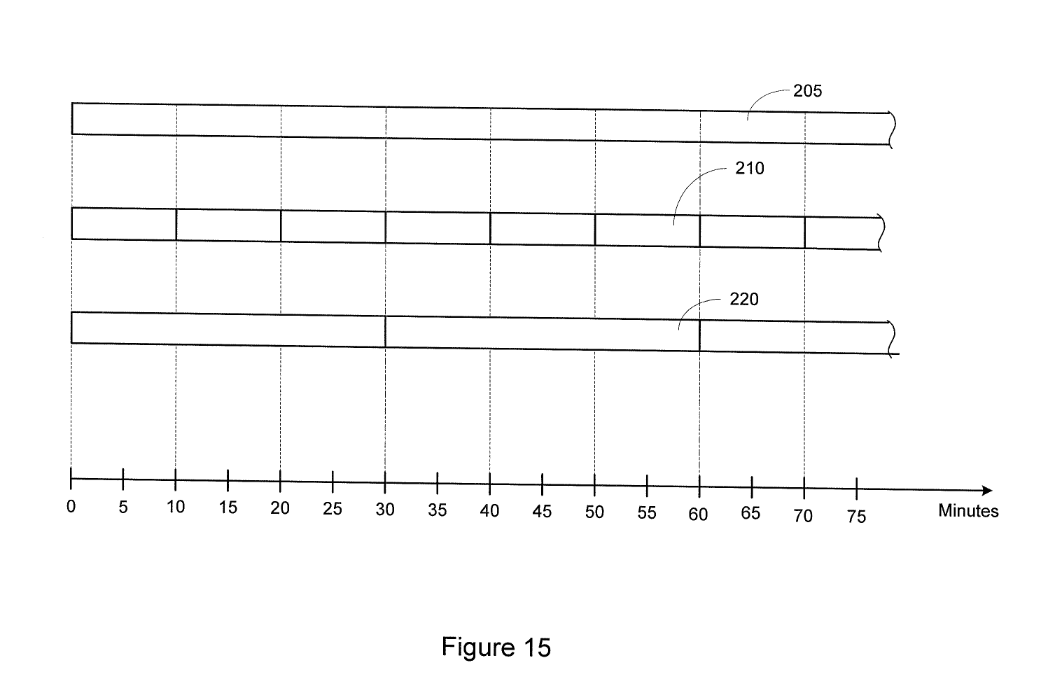

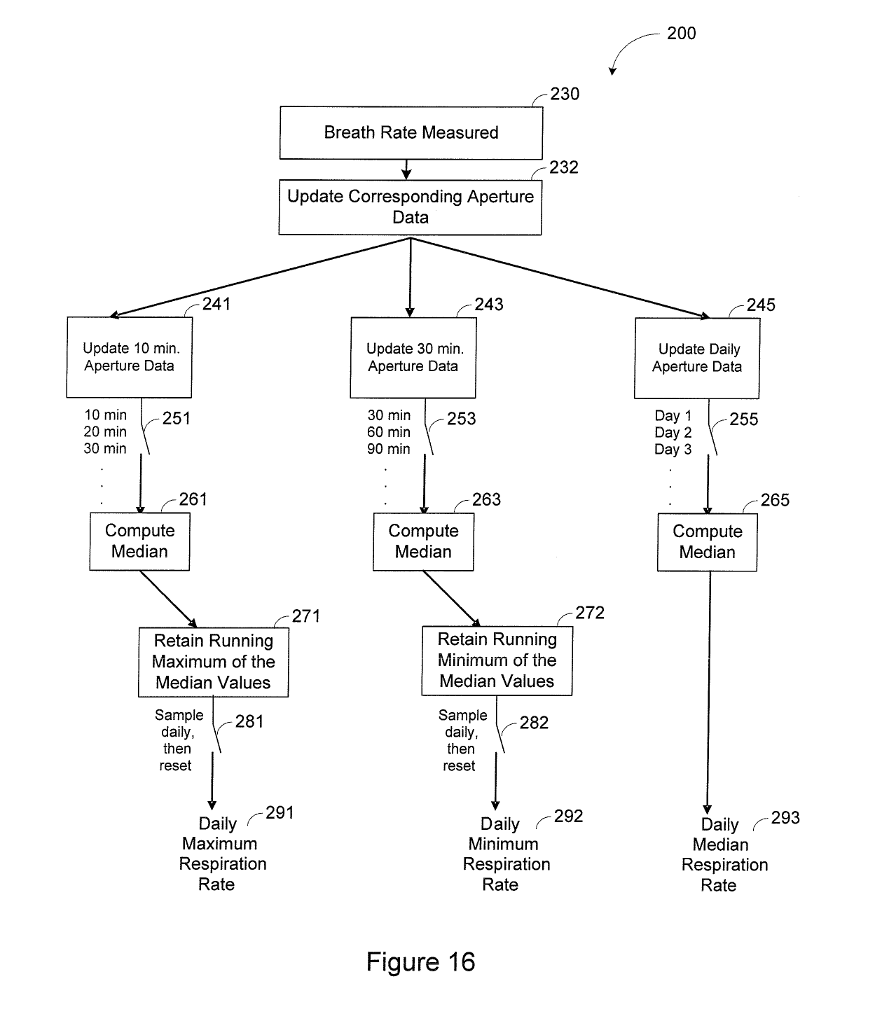

FIGS. 15 and 16 illustrate an implementation for determining respiration metrics including daily maximum respiration rate, daily median respiration rate, and daily minimum respiration rate in accordance with embodiments of the invention. Patient respiration is sensed and a respiration signal is generated. Overlapping apertures, as illustrated in FIG. 15, are superimposed on the respiration signal. The apertures include a 24 hour aperture 205 which is used to determine a daily median respiration rate. The apertures also include 10 minute apertures 210. The 10 minute apertures 210 are used to determine a daily maximum respiration rate. The apertures also include 30 minute apertures 220 which are used to determine a daily maximum respiration rate.

In one implementation, breath rates for each respiration cycle are measured and are used to determine median rates for an aperture. Several median rate processes are implemented, one corresponding to the median respiration rate of the 10 minute apertures, another corresponding to the median respiration rate of 30 minute apertures, and a third corresponding to a 24 hour median respiration rate. The daily minimum rate is determined from the median values of the 30 minute apertures 220 that span a 24 hour period. The daily maximum rate is determined from the median values of the 10 minute apertures 210 that span the 24 hour period. The daily median rate is the median value of the 24 hour period aperture 205. A process 200 for determining these daily metrics in accordance with one embodiment is illustrated in FIG. 16.

Breath rates are measured 230 from the respiration signal and used to acquire a daily minimum respiration rate, daily maximum respiration rate, and daily median respiration rate. The respiration signal may be generated, for example, by a transthoracic impedance sensor signal implemented in an implantable device, such as an implantable cardiac pacemaker or defibrillator. Breath detections received from the sensor may be pre-processed to avoid the use of spurious breath detections in determining the respiration metrics or trends. The process 200 may require that the breath rates meet certain criteria. In addition to providing breath rates for use in the respiration metric process 200, the respiration sensor circuitry, e.g., transthoracic impedance sensor, may provide data quality/status flags. Flags produced by the impedance sensor noise detection hardware/software may be used by the respiration metric process 200 to avoid using potentially corrupted data flagged as too noisy by the sensor. Further, the breath rates used to update the aperture data may be constrained to fall within a certain range of breath rates, e.g., about 4 breaths/minute to about 65 breaths/minute.

The measured respiration rate for the breath cycle is used to update 232 the data for each corresponding aperture. Data for each of the concurrently running apertures is updated 241, 243, 245 based on the measured breath rate. In some implementations, the breath rates may be computed in breaths/minute and the spacing of the histogram bins is 1 breath/minute. After an aperture is concluded, the median rate value for the aperture is computed 261, 263, 265. If an insufficient number of breaths are detected during an aperture, e.g., fewer than 100 breaths, then the aperture may be labeled invalid and a median for that aperture may not be computed. Throughout the 24 hour period, the running maximum of the median rate values for the 10 minute apertures is retained 271 and the running minimum of the median rate values for the 30 minute apertures is retained 272.

After the 24 hour period is concluded 281, 282, the daily maximum rate is reported 291, and the daily minimum rate is reported 292. The daily median respiration rate is determined 265 as the median rate value of the 24 hour aperture and reported 293. The daily maximum, minimum, and median values are preferably stored in the implantable medical device, and/or may be telemetered to a remote device, displayed on a display, or otherwise accessed by a physician or others. Additional information regarding respiration rate measurements which may be implemented in conjunction with the processes described herein is provided in commonly owned U.S. Patent Application Publication 2007/0135725 and the applications identified in the Related Application section of this disclosure, all of which are incorporated herein by reference.

Although various examples described herein provide an alert generated in response to a rise in respiratory rate above a threshold, those skilled in the art will appreciate that alerts may alternatively be generated upon respiration rate decreasing below a threshold. Aspects of the invention involve comparison of physiological parameters to alert criteria and generating an alert when the physiological parameters are equal to or beyond the alert criteria. Those skilled in the art will appreciate that a parameter value that is beyond a threshold can be, in various scenarios, either a parameter value below a threshold or a parameter value above a threshold.

As previously described, the patient's respiration rate is particularly useful in determining patient status and/or the effectiveness of a prescribed therapy. In one embodiment, the readmission alert is based on the respiration rate. In some scenarios, it is advantageous to employ a multi-sensor approach for more detailed assessment of certain patients or disorders. To this end, respiration and one or more additional physiological signals or parameters may be sensed and used together to assess changes in patient status and/or therapy effectiveness. For example, trends of left ventricular (LV) function, heart rate variability, disordered breathing, intrathoracic impedance, transthoracic impedance, intracardiac impedance, heart sounds, pulmonary arterial pressure, central venous pressure, physiological response to activity, percent in bi-ventricular pacing, patient activity, weight, heart rate, and/or blood pressure may be useful in determining changes in patient status and therapy effectiveness, particularly for HF patients.

Patient information developed from a patient symptom questionnaire may be used. The patient questionnaire can be presented to the patient via the patient communicator (or via a PC or mobile agent communicatively coupled to the patient communicator or to a patient management system) on a periodic basis, such as daily or weekly. In some configurations, a patient communicator is equipped with a user interface, allowing the patient to respond to questions appearing on a display. The patient questionnaire may be programmed to acquire information regarding symptoms that are difficult to acquire automatically such as feelings of fatigue, depression, dyspnea, edema, and/or subjective information related to the patient's health, patient compliance with prescribed therapies, and/or other information useful in the analysis of patient status and therapy effectiveness.

In some embodiments, the readmission alert criteria may be dynamically modified based on the patient's status. For example, if the patient's parameter trends generally indicate a decline in patient status, the readmission alert criteria may be automatically modified by the alert module to be more sensitive to changes in patient status. On the other hand, if the patient's physiological parameters generally indicate an improvement in overall health status, the readmission alert criteria may be automatically modified by the alert module to be less sensitive to changes in patient status. This feature automatically reconfigures the readmission alert criteria to avoid overburdening the patient's physician with unnecessary alerts.

In one embodiment, assessment of changes in therapy and/or need for optimization of therapy is based on a single parameter, such as respiration rate. The readmission alert criteria are met when the respiration rate metrics exceed a threshold(s), preferably for a predetermined period of time. When the alert criteria are met, this indicates a decline in the patient's condition and the readmission alert signal is generated.

When multiple parameters are tracked, the alert criteria may be based on relationships between the various parameters. For example, if both the respiration rate and LV function are used, then the readmission alert signal may be triggered if both parameters meet or exceed an alert threshold. In an alternative configuration, the readmission alert signal may be triggered if only one parameter meets or exceeds the alert threshold. In yet another configuration, the readmission alert signal may be triggered if one parameter meets or exceeds the readmission alert threshold and the other parameter is trending downward, indicating a worsening patient status. In another configuration, the readmission alert may be generated based on a different algorithm, such as a linear regression model, fuzzy logic, neural network, etc.

In certain embodiments, one parameter may be used to automatically alter the readmission alert threshold of another. This technique provides automatic adjustment in the sensitivity of the readmission alert. One approach involves using historic data of a parameter to adjust its own readmission threshold. Another approach involves one parameter affecting the threshold of another. For example, an activity parameter may be lower than normal level (i.e., the patient is not moving at all), while MaxRR is not reduced. This situation can result in lowering of the MaxRR alerting threshold.

By way of further example, if the patient's reports of dyspnea or tachypnea indicate this parameter is trending higher, then the threshold for the respiration rate may be adjusted downward so that a lower respiration rate will trigger the readmission alert. This threshold adjustment for the readmission alert criteria allows the readmission alert to be more responsive to the patient's perception of breathlessness, even when the respiration rate may not indicate a change that, when viewed in isolation, would indicate a problem.

In some embodiments the baseline value of the respiration rate may be learned automatically by the device. For example, during an initialization phase, system may make measurements of respiration rate to determine the baseline respiration rate for the patient. The period of time and frequency of measurements used to determine the baseline can be programmable. The alert threshold, in either breaths per minute over the baseline or percentage over the baseline, can be determined input by the physician or automatically determined by the system.

In some embodiments, the readmission alert module may take into account various contextual factors that have an impact on the physiological parameter used to generate the readmission alert. Additional sensors may be used to acquire information which provides a context for detected changes. For example, the patient's respiration rate depends directly on the patient activity. In one scenario, the patient's overall respiration rate may trend upward because he or she has embarked on a new exercise regimen. Without taking the patient's activity level into consideration, an unwarranted readmission alert may be produced. As another example, if the patient is sick, e.g., has pneumonia or other respiratory illness, then the effects of the illness may temporarily cause an increase in the patient's respiration rate. Optimization of therapy may not necessarily be indicated as a response to a temporary illness. Thus, the readmission alert module may take into account the patient's health status in determining whether to generate the readmission alert signal.

A readmission alert signal may be used for various purposes. In one embodiment, the readmission alert signal triggers a communication transmitted to the patient's physician or other health care provider. For example, the communication may involve an email, a telephone message, a fax and/or other type of communication directed to the patient's physician informing the physician of the detected change in therapy effectiveness and/or the need for therapy optimization or other intervention. The communication may range from cryptic indication of the change to a multi-level alert that indicates and/or provides an evaluation of the criticality of the change in patient status and/or need for therapy optimization. In some embodiments, the communication may provide additional information about the patient's status. For example, the communication may request that the physician log into the patient management server or the patient's website to view an update on the patient's status.

In some embodiments, the readmission alert signal may trigger an analysis of the patient's therapy. The analysis of patent therapy may make use of information used to generate the readmission alert along with other sensed physiological signals and/or other information. If an analysis of the therapy is performed, the communication to the patient's physician may provide suggestions for modification of the patient's therapy, such as by modifying a prescribed pharmacological therapy and/or by modifying device programming, e.g., re-programmed cardiac pacing parameters. In some implementations, the communication may indicate the need for a change in the type of device the patient is using. For example, the readmission alert module may analyze physiological parameters to determine if a patient needs a device that is capable of providing cardiac resynchronization therapy (CRT) by bi-ventricular and/or biatrial cardiac pacing. If the analysis concludes that CRT is indicated, the communication may include such a recommendation which may require a change in device type.

In some embodiments, the readmission alert signal may trigger an automatic or semi-automatic optimization of therapy. For example, optimization of therapy for HF patients implanted with CRT devices may involve optimizing various parameters of CRT.

CRT, through cardiac pacing, changes the electrical activation sequence of the heart by delivery of pacing pulses to multiple heart chambers. Modification of the electrical activation sequence changes the mechanical contractile sequence of the heart. If effective, the CRT improves the patient's hemodynamic status. CRT parameter optimization may analyze physiological signals and return parameters for CRT optimization based on the analysis of the physiological signals. Parameters for CRT returned by CRT optimization processes may include one or more cardiac pacing parameters such as atrioventricular delay (AVD), interventricular delay (IVD), interatrial delay (IAD), intersite pacing delays, pacing mode, tracking or non-tracking operation, pacing sites, pacing rate limits, and/or other pacing parameters, and/or non-pacing parameters, such as titrating the drugs being taken by the patients. CRT optimization methodologies may reduce the number of CRT recipients who have a less favorable response to CRT, through selecting the most appropriate cardiac pacing parameters.

The physiological signals used for CRT optimization may include cardiac electrical signals including cardiac signals sensed internal to the heart, denoted electrograms (EGMs). From EGMs, the heart's electrical activation sequence can be determined. The EGM may show excessive delays and/or blockages in portions of the heart's electrical conduction system. Exemplary CRT optimization processes based on analysis of cardiac electrical signals are described in commonly owned U.S. Pat. Nos. 7,013,176, 7,113,823, 7,181,285, 7,310,554, and 7,389,141, which are incorporated herein by reference.

Physiological signals used for CRT optimization may include signals associated with the heart's mechanical contractile sequence. In one example, heart sounds, or generally energies resulting from the heart's mechanical vibrations, indicate the mechanical contractile sequence. One particular type of heart sound, known as the third heart sound, or S3, has been found to be associated with heart failure. For example, an increase in S3 activity may indicate elevated filling pressures which may result in the state of decompensated heart failure. S3 amplitude is related to the filling pressure of the left ventricle during diastole. The pitch, or fundamental frequency, of S3 is related to ventricular stiffness and dimension. Chronic changes in S3 amplitude may be correlated to left ventricular chamber stiffness and degree of restrictive filling. An exemplary CRT optimization process based on analysis of heart sounds is described in commonly owned U.S. Patent Application Publication 2004/0127792 and U.S. Pat. No. 7,115,096 which are incorporated herein by reference.

Physiological signals used for CRT optimization may include heart rate from which heart rate variability data may be derived. Heart rate variability (HRV) is the beat-to-beat variability in heart rate. The main component of HRV is respiratory sinus arrhythmia (RSA). Under resting conditions, the healthy individuals exhibit periodic variation in beat to beat intervals with respiration. The heart rate accelerates during expiration and slows during inspiration. Reduction in HRV is a symptom of HF and is related to compromised neuro-hormonal status. An exemplary CRT optimization process based on analysis of HRV is described in commonly owned U.S. Pat. No. 7,343,199 which is incorporated herein by reference.

Physiological signals used for CRT optimization may include blood pressure signals which are directly related to hemodynamic status. In various examples, blood pressure may be sensed invasively or non-invasively and used to determine CRT parameters. For example, arterial pressure may be measured invasively by placing a pressure catheter in an artery, such as the radial artery. Left ventricular pressure may be measured via a pressure sensor inserted into the left ventricle. Non-invasive measurement of arterial pressure may be performed using a tonometer, phonocardiogram, or other methods. Pressure measurements obtained using these processes, or other processes, may be used to determine CRT parameters. Exemplary CRT optimization processes based on analysis of pressure signals are described in commonly owned U.S. Pat. Nos. 6,666,826, 7,158,830, and 7,409,244 which are incorporated herein by reference.

Other exemplary CRT optimization processes that may be used in conjunction with the methods and systems of the present invention are described in U.S. Pat. No. 7,206,634 which describes therapy optimization based on the use of mechanical sensors, U.S. Pat. No. 7,041,061 which describes therapy optimization based on quantification of wall motion asynchrony using echocardiographic images, U.S. Pat. No. 7,228,174 which describes therapy optimization based on impedance measurements, and U.S. Pat. No. 6,832,113, which describes therapy optimization based on a plethysmogram signal, all of which are incorporated herein by reference.

One or more of the above-referenced CRT optimization processes may be triggered by the readmission alert signal. In some embodiments, the pacing parameters returned from the CRT optimization processes may be automatically implemented to optimize the CRT therapy. Alternatively, the CRT parameters returned from the CRT optimization processes may be presented to the physician as recommended device programming changes. The physician may select the pacing parameters used to optimize CRT. In some embodiments, re-programming the device may be performed remotely by the physician.

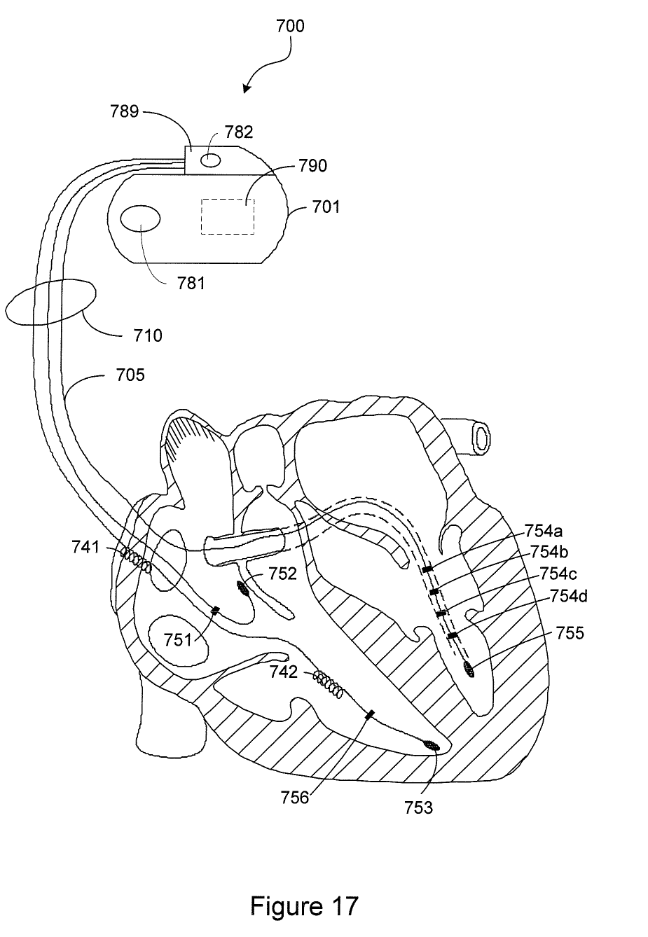

FIG. 17 illustrates a partial view of a patient implantable medical device that may be used to implement processes for detecting acute worsening of a patient's heart failure status and readmission alert generation in accordance with embodiments of the invention. The therapy device 700 illustrated in FIG. 17 may be used to acquire physiological data from which parameter trends may be developed for assessing changes in patient status and/or effectiveness of therapy. The therapy device 700 includes CRM circuitry enclosed within an implantable housing 701. The CRM circuitry is electrically coupled to an intracardiac lead system 710. Although an intracardiac lead system 710 is illustrated in FIG. 17, various other types of lead/electrode systems may additionally or alternatively be deployed. For example, the lead/electrode system may comprise and epicardial lead/electrode system including electrodes outside the heart and/or cardiac vasculature, such as a heart sock, an epicardial patch, and/or a subcutaneous system having electrodes implanted below the skin surface but outside the ribcage.

Portions of the intracardiac lead system 710 are shown inserted into the patient's heart. The lead system 710 includes cardiac pace/sense electrodes 751-756 positioned in, on, or about one or more heart chambers for sensing electrical signals from the patient's heart and/or delivering pacing pulses to the heart. The intracardiac sense/pace electrodes 751-756, such as those illustrated in FIG. 17, may be used to sense and/or pace one or more chambers of the heart, including the left ventricle, the right ventricle, the left atrium and/or the right atrium. The CRM circuitry controls the delivery of electrical stimulation pulses delivered via the electrodes 751-756. The electrical stimulation pulses may be used to ensure that the heart beats at a hemodynamically sufficient rate, may be used to improve the synchrony of the heart beats, may be used to increase the strength of the heart beats, and/or may be used for other therapeutic purposes to support cardiac function consistent with a prescribed therapy.

The lead system 710 includes defibrillation electrodes 741, 742 for delivering defibrillation/cardioversion pulses to the heart. The left ventricular lead 705 incorporates multiple electrodes 754a-754d and 755 positioned at various locations within the coronary venous system proximate the left ventricle. Stimulating the ventricle at multiple locations in the left ventricle or at a single selected location may provide for increased cardiac output in a patients suffering from HF, for example, and/or may provide for other benefits. Electrical stimulation pulses may be delivered via the selected electrodes according to a timing sequence and output configuration that enhances cardiac function. Although FIG. 17 illustrates multiple left ventricle electrodes, in other configurations, multiple electrodes may alternatively or additionally be provided in one or more of the right atrium, left atrium, and right ventricle. Optimization of CRT may involve selecting electrodes used to deliver pacing therapy.

Portions of the housing 701 of the implantable device 700 may optionally serve as one or more multiple can 781 or indifferent 782 electrodes. The housing 701 is illustrated as incorporating a header 789 that may be configured to facilitate removable attachment between one or more leads and the housing 701. The housing 701 of the therapy device 700 may include one or more can electrodes 781. The header 789 of the therapy device 700 may include one or more indifferent electrodes 782. The can 781 and/or indifferent 782 electrodes may be used to deliver pacing and/or defibrillation stimulation to the heart and/or for sensing electrical cardiac signals of the heart.

The cardiac electrodes can be used in conjunction with appropriate circuitry 790 disposed within the housing 701 of the therapy device 700 to sense transthoracic impedance and to develop a respiration signal from the transthoracic impedance measurements. As previously discussed, various respiration parameters can be determined from the respiration signal and a trend of the respiration parameter developed, although these processes may or may not be implemented by the therapy device 700. The respiration parameter is used to assess changes in therapy effectiveness or patient status.

In some embodiments, the therapy device 700 may also include sensors and/or circuitry for determining additional physiological parameters that may be useful in assessing therapy effectiveness. For example, the therapy device 700 may include an accelerometer used for sensing patient activity, may include circuitry for determining heart rate variability from the electrogram signal, may include circuitry to detect disordered breathing episodes, and/or may include circuitry for sensing various other parameters.

Communications circuitry is disposed within the housing 701 for facilitating communication between the CRM circuitry and a patient-external device, such as an external programmer or patient communicator coupled to a patient management server. In some embodiments the therapy device may include a sensor configured to sense the metabolic need so that the pacing rate can be adapted to accommodate the patient's metabolic need.

In certain embodiments, the therapy device 700 may include circuitry for detecting and treating cardiac tachyarrhythmia via defibrillation therapy and/or anti-tachyarrhythmia pacing (ATP). Configurations providing defibrillation capability may make use of defibrillation coils 741, 742 for delivering high energy pulses to the heart to terminate or mitigate tachyarrhythmia.

CRM devices using multiple electrodes, such as illustrated herein, are capable of delivering pacing pulses to multiple sites of the atria and/or ventricles during a cardiac cycle. Certain patients may benefit from activation of parts of a heart chamber, such as a ventricle, at different times in order to distribute the pumping load and/or depolarization sequence to different areas of the ventricle. A multi-electrode pacemaker has the capability of switching the output of pacing pulses between selected electrode combinations within a heart chamber during different cardiac cycles.

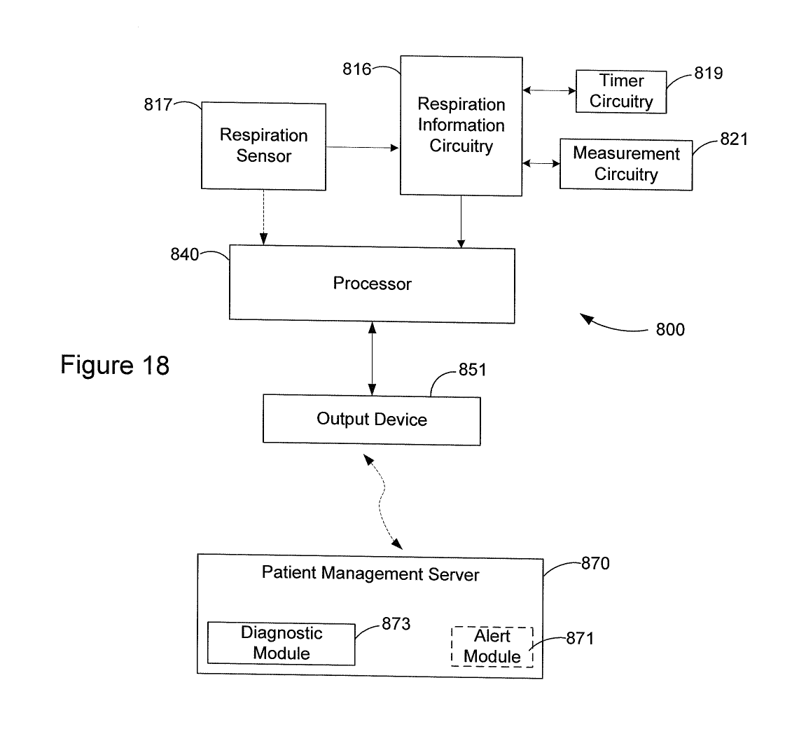

FIG. 18 is a block diagram of a medical system 800 that may implement various diagnostic, readmission alert generation, and/or therapy processes in accordance with various embodiments. In general terms, the system 800 shown in FIG. 18 is particularly well suited for assessing a patient's heart failure status and, more particularly, for detecting onset of acute worsening of a patient's heart failure condition and generating a readmission alert in accordance with embodiments of the invention. The system 800 is preferably configured to implement respiration rate trending techniques as are described herein and for generating a hospital readmission alert signal.

According to various embodiments, the medical system 800 includes a respiration sensor 817 configured to generate a signal indicative of patient respiration. As previously discussed, the respiration sensor 817 may be an implantable sensor or an external sensor (or a sensor that combines implantable and external components). The system 800 includes respiration information circuitry 816 coupled to the respiration sensor 817 and configured to make various respiration measurements using the signal indicative of patient respiration provided by the respiration sensor 817. The respiration information circuitry 816 is preferably configured to measure a median respiration rate (MedRR) and a maximum respiration rate (MaxRR), and may optionally be configured to measure a minimum respiration rate (MinRR).

FIG. 18 shows timer circuitry 819 and measurement circuitry 821 respectively coupled to respiration information circuitry 816. Timer circuitry 819 is configured to time a plurality apertures in a manner discussed hereinabove. The measurement circuitry 821 is configured to measure a respiration rate for each breath cycle of each aperture, estimate a respiration rate for each aperture based on the measured respiration rates, and determine MedRR and MaxRR from the estimated respiration rates in a manner previously described above.

The processor 840 is coupled to the respiration information circuitry 816 and configured to determine whether an abnormality exists in MedRR and whether an abnormality exists in MaxRR, preferably in a manner previously described. The processor 840 may further be configured to determine whether an abnormality exists in MinRR. An output device 851 is coupled to the processor 840 and configured to generate an output indicative of the patient's tachypnea status in response to determining the abnormality in MedRR and MaxRR.

The processor 840 may be configured to determine whether there is an abnormality in MinRR, and the output device 851 may be configured to generate an output indicative of the patient's heart failure status based on the abnormality determination in MedRR, MaxRR, and MinRR. The processor 840 may be configured to determine the patient's heart failure status by determining whether abnormality (e.g., elevation) in MedRR, MaxRR, and MinRR are within a predetermined range of abnormality, and may further cooperate with the output device 851 to generate an output indicating that the patient's heart failure status may be worsening.

The processor 840 and the output device 851 may be implemented to cooperatively generate an output indicative of the patient's heart failure status based at least in part on the processor 840 determining whether a near-term measure of MinRR is greater than a baseline for MedRR. The processor 840 and the output device 851 may be implemented to cooperatively generate an output indicative of the patient's heart failure status based at least in part on the processor 840 determining whether a near-term measure of MedRR is greater than a baseline for MaxRR.

The output device 851 may be configured to produce a readmission alert in response to conditions described above, and communications circuitry of the system may be configured to communicate the alert to a networked patient management system 870 or other receiving device or system (e.g., communicator or PC). The patient management system 870 may include an alert module 871 and a diagnostic module 873 for implementing a respiration rate trending and readmission alert algorithm in accordance with various described embodiments of the present invention. As previously discussed, functions performed by the alert module 871 and/or the diagnostic module 873 may alternatively be implemented by the processor 840 or other component(s) of the medical system 800.

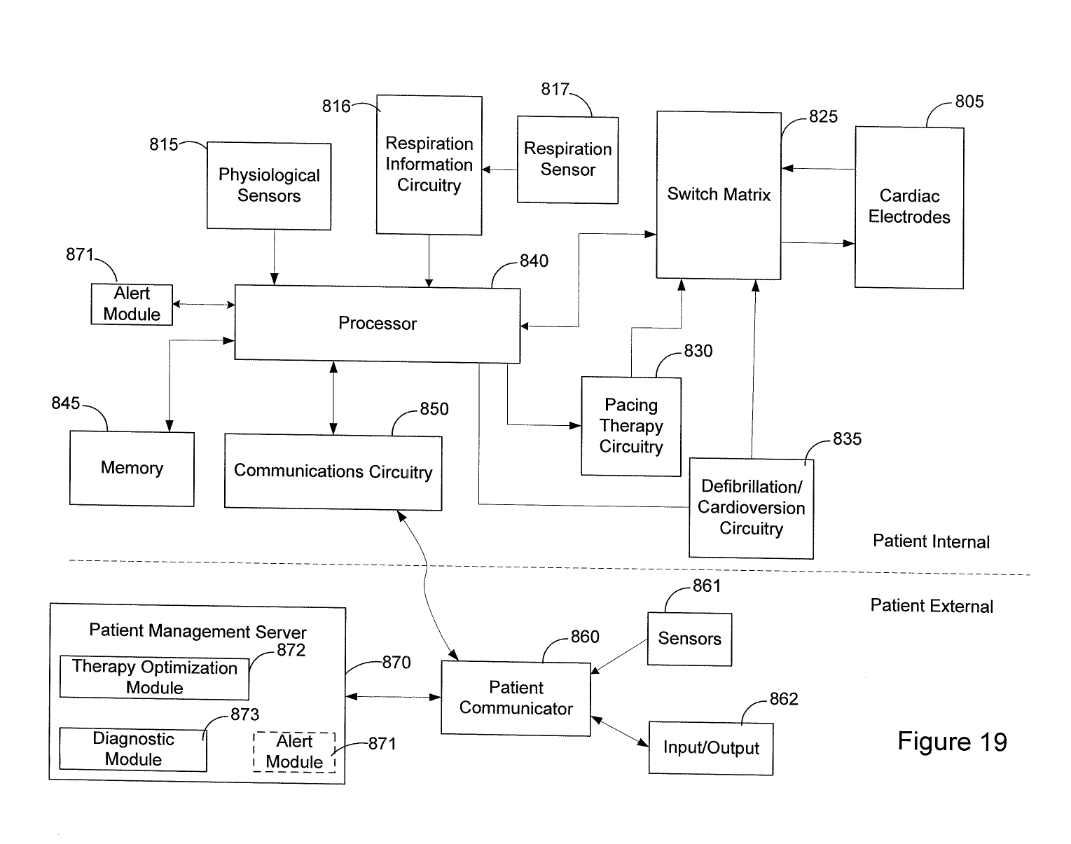

FIG. 19 is a block diagram of one embodiment of a medical system 800 that may be configured to implement respiration rate trending and readmission alert generation processes in accordance with various embodiments of the present invention. The system 800 includes a patient internal device (implantable CRM device) that incorporates pacing therapy circuitry 830 configured to deliver pacing pulses to a heart via cardiac electrodes 805. The implantable CRM device may optionally include defibrillation/cardioversion circuitry 835 configured to deliver high energy defibrillation or cardioversion stimulation to the atria or ventricles of the heart for terminating tachyarrhythmias.

The electrodes 805 are coupled to switch matrix 825 circuitry used to selectively couple electrodes 805 to other components of the CRM device. The electrodes 805 may be used in conjunction with respiration sensor 816 (e.g., transthoracic impedance circuitry) to sense the patient's respiration signal. Additional physiological sensors 815 may also be included in the CRM device.

The processor 840 controls the therapy and sensing operations of the CRM device. Additionally, the processor 840 manages data storage operations to allow storage in memory 845 signals, parameter measurements and/or parameter trends developed using the respiration sensor data and, if used, the data from the other physiological sensors 815. The processor 840 preferably implements respiration rate trending and readmission alert logic as described herein for determining a patient's tachypnea and/or heart failure status, such as early onset of worsening of a patient's heart failure. In some automatic configurations, the CRM device may include the readmission alert module that assesses changes in therapy effectiveness and generates the readmission alert signal based on these changes. Additionally or alternatively, the CRM device may include diagnostic or therapy modification circuitry. The diagnostic circuitry may assess the parameter trends stored in memory to diagnose a disease or to assess the progression of a disease or symptoms associated with the disease. Responsive to a signal generated by the readmission alert module, the processor 840 may automatically initiate a therapy optimization procedure.

A CRM device typically includes a battery power supply (not shown) and communications circuitry 850 for communicating with the external patient communicator 860, device programmer (not shown) or other patient-external device. Data stored in the memory of the CRM device, such as signals, measurements or trends from the respiration signal and/or other physiological sensor signals, can be transferred from the memory 845 of the CRM device to the patient communicator 860 via the communications circuitry 850. Transfer of this information may be performed periodically, on demand, or in response to a triggering event.

In some embodiments, the patient communicator 860 receives the information from the CRM device and forwards it to the patient management server 870 for assessment of changes in a patient's tachypnea and/or heart failure status and/or therapy effectiveness. The patient management 870 server may optionally include a readmission alert module 871, configured to analyze the information received from the CRM device via the patient communicator. The readmission alert module 871 is configured to generate readmission alert signals based on comparison of parameters to alert criteria. The patient management server 870 may optionally include a diagnostic module 873 for diagnosing a disease presence and/or monitoring the progression, regression, or status quo of a disease condition. The patient management server 870 may optionally include a therapy optimization module 872 configured to evaluate the patient's condition and assess therapy settings based on the parameter information received from the CRM device. After analysis, modification of therapy parameters may be transferred to the CRM device to automatically effect changes in the patient's therapy in some implementations.

In some embodiments, the patient communicator 860 may also include circuitry and/or software to make parameter measurements and develop parameter trends. The alert module, therapy optimization module, and/or diagnostic module may be fully or partially disposed in the patient communicator 860 imbuing the patient communicator 860 with partial or full functionality to analyze the parameter values, develop parameter trends, assess changes in patient status and/or therapy effectiveness, and determine appropriate therapy adjustments. In this configuration, the patient communicator 860 may make recommendations for therapy optimization and/or download optimized therapy parameters to the CRM device, and/or trigger the CRM device to implement processes for determining optimized parameters.

The patient communicator 860 may be coupled to various sensors 861 for acquiring information about patient parameters, e.g., patient externally acquired parameters, in addition to the implantably acquired respiration parameters. In certain embodiments, the sensors 861 may include a blood pressure sensor and weight scale. The sensors 861 and patient communicator 860 may employ wireless communication technology such as Blue Tooth, or other RF telemetry protocols. The patient may access the sensors 861 in accordance with a prescribed testing schedule. For example, the patient may measure his or her weight and blood pressure at periodic intervals and this information may be communicated from the sensors 861 to the patient communicator 860.

The patient communicator 860 may be coupled to an input/output device 862 including a keyboard, pointing device, touch panel or other input device, and a display. The patient may interact the input/output device to answer questionnaires displayed to the patient on the display. The patient's answers to the questions may be trended along with the measurements acquired from the sensors 815, 816 coupled to the CRM device or sensors 861 coupled to the patient communicator 860. The additional parameters may be used along with the respiration parameters to assess changes in therapy effectiveness.

The components, functionality, and structural configurations depicted herein are intended to provide an understanding of various features and combination of features that may be incorporated in an implantable or patient-external medical device or system. For example, an external respiration sensor or other physiologic sensor may be used to acquire patient physiologic information, and an external processor or other logic device may be employed to compute respiration metrics or other physiologic data metrics, and generate a readmission alert based on detection of an acute deleterious change in such metrics. It is understood that a wide variety of such device or system configurations are contemplated, ranging from relatively sophisticated to relatively simple designs. As such, particular implantable/external or cardiac monitoring and/or stimulation device configurations may include particular features as described herein, while other such device configurations may exclude particular features described herein.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

1. A method, comprising:
collecting respiration data for a heart failure patient at least during hospitalization and a time after discharge of the heart failure patient from a hospital;
measuring, using the collected respiration data, a median respiration rate (MedRR) and a maximum respiration rate (MaxRR);
analyzing the MedRR and MaxRR for a change in the MedRR and MaxRR;
detecting further deterioration of the patient's heart failure status based at least in part on detecting an acute deleterious change in the MedRR and MaxRR indicative of an abnormality in MedRR and MaxRR; and
generating a readmission alert in response to detecting the acute deleterious change in the MedRR and MaxRR, the readmission alert indicating that the further deterioration in the patient's heart failure status may require readmission of the patient to the hospital.

2. The method of claim 1, wherein:
detecting further deterioration of the patient's heart failure status comprises:
determining whether the acute deleterious change in the MedRR exceeds a first threshold; and
determining whether the acute deleterious change in the MaxRR exceeds a second threshold; and
generating the readmission alert comprises generating the readmission alert in response to the acute deleterious change in the MedRR and MaxRR exceeding the first and second thresholds, respectively.

3. The method of claim 2, wherein:
the first threshold is developed using the MedRR collected before discharge of the heart failure patient from the hospital;
determining whether the acute deleterious change in the MedRR exceeds the first threshold is based on a comparison of at least an MedRR collected after discharge of the heart failure patient from the hospital relative to the MedRR collected before discharge;
the second threshold is developed using the MaxRR collected before discharge of the heart failure patient from the hospital; and
determining whether the acute deleterious change in the MaxRR exceeds the second threshold is based on a comparison of at least a MaxRR collected after discharge of the heart failure patient from the hospital relative to the MaxRR collected before discharge.

4. The method of claim 2, wherein each of the first and second thresholds is developed using respiration data collected during a specified period of time prior to patient discharge from the hospital, and the acute deleterious change in the MedRR and MaxRR is detected within about one day to about two months after patient discharge from the hospital.

5. The method of claim 2, wherein each of the first and second thresholds is developed using respiration data collected during a specified period of time prior to patient discharge from the hospital, and the acute deleterious change in the MedRR and MaxRR is detected within about one day to about one month after patient discharge from the hospital.

6. The method of claim 1, comprising:
developing a baseline and a threshold for each of the MedRR and MaxRR using respiration data collected during hospitalization of the heart failure patient;
measuring the MedRR and the MaxRR after discharge of the heart failure patient from the hospital; and
detecting further deterioration of the patient's heart failure status is based at least in part on detecting an acute deleterious change in the post-discharge MedRR and MaxRR relative to their respective baselines and thresholds.

7. The method of claim 6, comprising receiving a discharge notification associated with a time of heart failure patient discharge from the hospital, and using the discharge notification to distinguish between respiration data collected during hospitalization and respiration data collected after discharge of the heart failure patient from the hospital.

8. The method of claim 1, comprising:
collecting patient activity data for the patient; and
generating the readmission alert in response to the acute deleterious change in the MedRR and MaxRR and the patient activity data indicating that the patient is not engaged in patient activity.

9. The method of claim 1, comprising:
measuring, using the respiration data, a minimum respiration rate (MinRR);
analyzing the MinRR for a change in the MinRR; and
generating the readmission alert in response to the acute deleterious change in the MedRR and MaxRR and a substantially non-elevated MinRR.

10. The method of claim 1, wherein collecting, analyzing, detecting, and generating processes are performed by a medical device implanted in the patient.

11. The method of claim 1, comprising communicating the readmission alert to a network server for access by a clinician.

12. A method, comprising:
collecting physiologic data for a heart failure patient at least during hospitalization and a time after discharge of the heart failure patient from a hospital;
analyzing the collected physiologic data for a change in the collected physiologic data;
detecting further deterioration of the patient's heart failure status based at least in part on detecting an acute deleterious change in the collected physiologic data indicative of an abnormality in one or more physiologic parameters represented by the physiologic data; and
generating a readmission alert in response to the acute deleterious change in the collected physiologic data, the readmission alert indicating that the further deterioration in the patient's heart failure status may require readmission of the patient to the hospital.

13. The method of claim 12, comprising:
developing one or more baselines and thresholds for physiologic data collected during hospitalization of the heart failure patient;
analyzing, using physiologic data collected after discharge of the heart failure patient from the hospital, the collected physiologic data for a change in the post-discharge collected physiologic data; and
detecting further deterioration of the patient's heart failure status is based at least in part on detecting an acute deleterious change in the post-discharge collected physiologic data relative to the one or more baselines and thresholds.

14. The method of claim 13, comprising receiving a discharge notification associated with a time of heart failure patient discharge from the hospital, and using the discharge notification to distinguish between physiologic data collected during hospitalization and physiologic data collected after discharge of the heart failure patient from the hospital.

15. The method of claim 13, wherein the one or more thresholds are developed using physiologic data collected during a specified period of time prior to patient discharge from the hospital, and the acute deleterious change in the post-discharge collected physiologic data is detected within about one day to about two months after patient discharge from the hospital.

16. The method of claim 13, wherein the one or more thresholds are developed using physiologic data collected during a specified period of time prior to patient discharge from the hospital, and the acute deleterious change in the post-discharge collected physiologic data is detected within about one day to about one months after patient discharge from the hospital.

17. The method of claim 12, wherein collecting, analyzing, detecting, and generating are performed by a medical device implanted in the patient.

18. The method of claim 12, comprising communicating the readmission alert to a network server for access by a clinician.

19. The method of claim 12, wherein detecting further deterioration of the patient's heart failure status comprises comparing the change in the collected physiologic data to one or more thresholds, the one or more thresholds comprising at least one of a predetermined change in the collected physiologic data, a predetermined rate of change in the collected physiologic data, and a predetermined variance in the collected physiologic data.

20. The method of claim 12, wherein the physiologic data comprises at least one of respiration data, respiration metrics, blood pressure data, intra-abdominal pressure sensor, heart sounds data, heart rate variability data, lead impedance data, electrogram data, electrocardiogram data, electrical activation sequence data, cardiac vector axis data, QRS axis data, mechanical sensor data, mechanical contractile sequence data, vascular tone data, neuro-hormonal data, hemodynamic data, intrathoracic impedance data, transthoracic impedance data, intracardiac impedance data, heart sounds data, physiological response to activity data, pulmonary arterial pressure data, central venous pressure data, electro-mechanical delay data, patient weight data, patient posture data, and patient symptom questionnaire data.

21. A medical system, comprising:
a respiration sensor configured to generate a signal indicative of patient respiration;
respiration information circuitry coupled to the respiration sensor, the respiration information circuitry configured to collect respiration data for a heart failure patient and to measure a median respiration rate (MedRR) and a maximum respiration rate (MaxRR) using the collected respiration data;
a processor coupled to the respiration information circuitry, the processor executing program instructions that configure the processor to:
cause the respiration information circuitry to collect the respiration data for the heart failure patient during hospitalization and after discharge of the heart failure patient from a hospital;
analyze the MedRR and MaxRR for a change in the MedRR and MaxRR and;
detect further deterioration of the patient's heart failure status based at least in part on detecting an acute deleterious change in the MedRR and MaxRR indicative of an abnormality in MedRR and MaxRR; and
an output device coupled to the processor and configured to generate a readmission alert in response to detection of the acute deleterious change in the MedRR and MaxRR, the readmission alert indicating that the further deterioration in the patient's heart failure status may require readmission of the patient to the hospital.

22. The system of claim 21, wherein the processor executes program instructions that configure the processor to perform any of the processes of claim 1 through claim 11.
